(19) United States
(12) Patent Application Publication
(10) Pub. No.: US 2007/0154981 A1
(43) Pub. Date: Jul. 5, 2007

(54) Insulin-producing cells derived from stem cells

(75) Inventors: Yuichi Hori, Chuo-Ku (JP); Seung Kim, Stanford, CA (US)

Correspondence Address: FISH & NEAVE IP GROUP;ROPES & GRAY LLP, ONE INTERNATIONAL PLACE, BOSTON, MA, 02110-2624, US

(73) Assignee: THE BOARD of TRUSTEES of the LELAND STANFORD JUNIOR UNIVERSITY, PALO ALTO, CA (US)

(21) Appl. No.: 10/545,581

(22) PCT Filed: Feb. 17, 2004
(86) PCT No.: PCT/US04/04681

§ 371 Date: Nov. 28, 2006

Related U.S. Application Data
(60) Provisional application No. 60/447,684, filed on Feb. 14, 2003.

(51) Int. Cl. C12P 21/06; C07H 21/04; C12N 5/08; C07K 14/62

(52) U.S. Cl. 435/69.1; 435/320.1; 435/325; 435/368; 530/303; 536/23.5

(57) ABSTRACT

The disclosure provides, among other things, insulin-producing cells derived from stem cells, such as human stem cells and neural stem cells. The disclosure discloses a relationship between caudalizing factors and the differentiation of insulin-producing cells.

### CROSS-REFERENCE TO RELATED APPLICATIONS

This application claims the benefit of the filing date of U.S. Provisional Patent Application Ser. No. 60/447,684 filed Feb. 14, 2003, entitled “Insulin-Producing Cells Derived from Stem Cells” and listing Seung Kim and Yuichi Hori as inventors. The aforementioned provisional application is incorporated herein in its entirety.

### BACKGROUND

Diabetes mellitus (DM) is a major cause of morbidity and mortality worldwide, and incidence rates of type I and type II DM are increasing. In type I DM, destruction of insulin-producing pancreatic islets leads to a prolonged illness often culminating in devastating multisystem organ failure and early mortality. Clinical trials demonstrate that tight glucose regulation can prevent the development of diabetic complications, but attempts to achieve this regulation by exogenous insulin administration are only partially successful.

Recent evidence suggests that islet cell transplantation with improved systemic immunosuppression may provide a short-term durable remission in insulin requirements in type I diabetics (Shapiro et al, 2000, N Engl J Med. 343: 230-238; Ryan et al, 2001, Diabetes 50: 710-719). However, in DM and the vast majority of other human diseases amenable to treatment by tissue replacement, there is an extreme shortage of engraftable donor tissues. An expandable source of tissues like human stem cells may provide the best promise for tissue replacement strategies for human diseases.

Stem cells, including embryonic stem (ES) cells and various adult stem cells provide a promising potential means for cell-replacement therapy in human diseases. Stem cells may provide serve as an inexhaustible source for the production of replacement islets for transplantation in diabetic humans. However, conditions to produce stably-differentiated functional insulin-producing cell compositions with stem cells generally have not been developed to a clinically satisfactory level.

Methods to provide a renewable source of replacement islets from stem cells could transform therapeutics in DM. Likewise, methods for stimulating the production of insulin-producing cells in patients could also have significant therapeutic effects. Additionally, improved in vitro systems that mimic islet cell development may be used as tools in, for example, drug discovery programs to identify DM therapeutics.

### SUMMARY

In certain aspects the disclosure provides pancreatic hormone-producing cells, and particularly insulin-producing cells, derived from human stem cells, such as human neural, neuroendocrine or embryonic stem cells. In certain aspects the disclosure provides methods for culturing stem cells in the presence of a neural/endoderm caudalizing factor to obtain cells that are responsive to an islet cell differentiation factor (ICDF). In certain aspects the disclosure provides methods for obtaining pancreatic hormone-producing cells, and particularly insulin-producing cells, by culturing ICDF-responsive cells in the presence of an ICDF. In certain aspects, the disclosure provides methods for making insulin producing cells by culturing stem cells successively in a medium comprising a neurallendoderm caudalizing factor and a medium comprising an ICDF. Cells produced according to the disclosed methods may be used for a variety of purposes, including amelioration of disorders associated with pancreatic insufficiency.

In certain aspects, the disclosure provides methods for making a cell composition comprising cells that are receptive to treatment with an islet cell differentiation factor (ICDF). In one embodiment, a method comprises culturing stem cells with a neural/endoderm caudalizing factor. Optionally, the stem cells are embryonic, neural or neuroendocrine stem cells, and preferably the stem cells are from a stem cell line. In a preferred embodiment, the stem cells are human stem cells. In a preferred embodiment, the stem cells are neural stem cells that are positive for binding to a monoclonal antibody AC133 or to a monoclonal antibody 5E12, or cells derived therefrom. In certain embodiments, neural/endoderm caudalizing factors for use with a disclosed method are caudalizing retinoic acid signaling activator, including, for example, retinoids and non-retinoids that act on the retinoid signaling pathway. Other caudalizing factors are described herein.

In certain aspects, the disclosure provides methods for making pancreatic hormone-producing cells such as insulin-producing cells. In one embodiment, a method comprising culturing stem cells, and particularly neural or neuroendocrine stem cells, in at least two different media, wherein at least one of said media comprises a neural/endoderm caudalizing factor. Certain methods for producing pancreatic hormone-producing cells comprise culturing stem cells with a neural/endoderm caudalizing factor, followed by culturing in the presence of an islet cell differentiation factor. Certain methods comprise culturing ICDF-responsive cells in the presence of an ICDF. Preferred ICDFs include nicotinamide, IGF-1, IGF-1 agonists, and butyric acid (and salts such as sodium butyrate). In certain embodiments, methods described herein result in the production of cell compositions that resemble pancreatic islets in that the cell compositions comprise two or more of the following cell types: insulin-producing cells, somatostatin producing cells, pancreatic polypeptide producing cells and glucagon producing cells. In preferred embodiments, pancreatic hormone producing cells are viable and non-apoptotic.

In certain embodiments, pancreatic hormone-producing cells produce only one of the following pancreatic hormones: insulin, glucagon, pancreatic polypeptide (PP) and somatostatin. Insulin-producing cells disclosed herein are preferably positive for one or more markers selected from the group consisting of: insulin (any of the various chains, including, for example, C-peptide, mature insulin or proinsulin), GLUT2, glucokinase, PDX-1, IAPP, SUR1, PC1/3, PC2 and KIR6.2. In preferred embodiments, pancreatic hormone producing cells are viable and non-apoptotic. Insulin-producing cells may be produced in a variety of cell composition forms, including, for example, cell clusters. Preferably at least 50% of the cells of a cell composition produce insulin. Preferably at least 50% of the cells of a cell composition are non-apoptotic. Pancreatic hormone producing cells may be non-proliferative.

In certain embodiments, pancreatic hormone-producing cells are derived from embryonic, neural or neuroendocrine stem cells, and particularly from an embryonic, neural or neuroendocrine stem cell line. Optionally, pancreatic hormone-producing cells derived from neural or neuroendocrine stem cells retain one or more characteristics of a neural or neuroendocrine cell.

In certain embodiments, the disclosure provides therapeutic cell composition comprising insulin-producing cells disclosed herein and a therapeutically acceptable excipient, such as a capsule, buffer or other excipient.

In certain embodiments, the disclosure provides methods for ameliorating, in a subject, a condition related to insufficient pancreatic function. A method may comprise administering to the subject an effective amount of insulin-producing cells of a type disclosed herein. In certain embodiments, the administered cells are derived from embryonic, neural or neuroendocrine stem cells. In certain embodiments, the subject is a human or optionally a non-human animal, and accordingly, the disclosure provides non-human animals comprising an insulin-producing cell composition of a type disclosed herein. The disclosure further provides methods, such as those described above, for preparing a cellular medicament for the treatment of a condition related to insufficient pancreatic function, such as a form of diabetes.

In certain embodiments, a method for ameliorating, in a subject, a condition related to insufficient pancreatic function, comprises: (a) obtaining from the subject or an HLA-matched donor a sample comprising neural or neuroendocrine stem cells; (b) culturing one or more of the neural or neuroendocrine stem cells in the presence of a neural/endoderm caudalizing factor to obtain a first cell composition; (c) culturing the first cell composition in the presence of an islet cell differentiation factor to obtain a second cell composition, wherein the second cell composition comprises insulin producing cells; and (d) administering to the subject an effective amount of insulin-producing cells. Optionally, the sample is cultured so as to enrich for and/or cause the proliferation of neural or neuroendocrine stem cells. The sample may be obtained, for example, from a tissue such as a tissue comprising cells of the peripheral nervous system, a tissue comprising cells of the central nervous system or a tissue comprising neuroendocrine cells. The sample may be obtained by, for example, trans-cranial biopsy, olfactory bulb biopsy, spinal cord biopsy or skin biopsy.

In certain aspects, the disclosure provides methods for assessing a test agent for islet cell differentiation factor activity. Certain method embodiments comprise contacting cells that are receptive to treatment with an islet cell differentiation factor with the test agent; and detecting an islet cell marker, wherein a test agent that stimulates the formation of cells expressing the islet cell marker has islet cell differentiation factor activity.

In certain aspects, the disclosure provides methods for testing the developmental potential of a cell of interest. In some embodiments, the method comprises co-culturing stem cells and one or more cells of interest through one or more culture conditions that cause the stem cells to give rise to insulin-producing cells, wherein at least one of the culture conditions include culturing in the presence of neural/endoderm caudalizing factor; and determining the identity of cells derived from the cell of interest, thereby testing the developmental potential of the cell of interest. Optionally, one of the culture conditions includes culturing in the presence of an islet cell differentiation factor. In certain embodiments, cells of interest may be cultured in the presence of a fraction of cells cultured according to a method of the disclosure. For example, cells of interest may be cultured in the presence of a soluble fraction obtained from stem cells that were cultured in the presence of a caudalizing factor.

In certain aspects, the disclosure provides methods for predicting the ability of an affinity reagent, such as an antibody, to bind to a pancreatic progenitor cell. In certain embodiments, a method involves screening a plurality of affinity reagents to identify those affinity reagents that bind to a cell that is in the process of developing into an pancreatic hormone producing cell. An affinity reagent that binds selectively to the cells prepared according to a method of the disclosure is likely to bind to a pancreatic progenitor cell. Optionally, the affinity reagent may be further tested for binding specificity in a tissue sample, such as a pancreatic sample or a sample from pre-pancreatic tissue.

The embodiments and practices of the present disclosure, other embodiments, and their features and characteristics, will be apparent from the description, figures and claims that follow, with all of the claims hereby being incorporated by this reference into this Summary.

### BRIEF DESCRIPTION OF THE DRAWINGS

FIG. 1: Sequence of extracellular signals regulating pancreatic development and islet differentiation (top row) compared to signals regulating formation of IPCCs from ES or NS cells (bottom row). RA=retinoic acid, SHH=Sonic hedgehog, LIF=leukemic inhibitory factor. Other signals and molecular markers ofpancreas endodermal cell fate are shown and described herein.

FIG. 2. Development of insulin-producing cell clusters from undifferentiated neurospheres (stage 1). Immunohistochemical detection of indicated markers in undifferentiated human neural stem cells (stage 1), or in cells exposed to retinoic acid (stage 2) then nicotinamide and IGF1 (stage 3). At stage 1, cells expressed nestin (brown cytoplasmic staining, contrasted by blue nuclear counterstain) and the proliferation antigen Ki67. Nestin expression was vitually extinguished at stages 2 and 3 when islet cell hormones like insulin, somatostatin, and pancreatic polypeptide are expressed. By stage 3, >90% of cells are no longer proliferating, as indicating by lack of Ki67 expression. Except for the nestin staining in the top 3 panels, signal intensity for a given marker is rendered on a gray-to-black scale.

FIG. 3 Immununohistochemical detection of insulin and other islet cell products in stage 3 NS-derived tissue. (Top row) Insulin expression appears green (revealed with a FITC-conjugated secondary antibody). 7AAD is a nuclear stain that helps reveal intact nuclear morphology in the majority of insulin+ cells at this stage. (Middle row) C-peptide expression appears red (revealed with a Cy3-conjugated secondary antibody) and is detected in all insulin+ cells. (Bottom row) TUNEL assay for apoptotic nuclei (red) shows that >95% of insulin+ cells at stage 3NI are not apoptotic.

FIG. 4. Expression of islet cell markers and β-cell markers in NS cell-derived IPCCs. Note that somatostatin+ and pancreatic polypeptide+ cells are distinct from insulin+ cells. The great majority of insulin+ cells do not have TUNEL+ nuclei and are therefore not apoptotic. Glucokinase is a key enzyme required for glucose sensing in β cells and expressed in all insulin+ cells in IPCCs. This expression, combined with observed exclusion of somatostatin and PP from insulin+ cells in IPCCs provides evidence that some mechanisms regulating production of pancreatic β-cells are recapitulated during in vitro differentiation of IPCCs. All images obtained by confocal microscopy of microtome-sectioned IPCCs.

FIG. 5. Insulin yield from isolated IPCCs derived from human neural stem cell cultures exposed to specific sequences of conditions and growth factors. Conditions: (1) 100 nM Retinoic Acid +30 nM Sonic Hedgehog for 2 weeks then 10 mM Nicotinamide+2 nM Activin A for 1 week; (2) 200 nM Retinoic Acid for 2 weeks then 10 mM Nicotinamide+2 nM Activin A for 1 week; (3) 100 nM Retinoic Acid+30 nM Sonic Hedgehog for 2 weeks then 10 mM Nicotinamide+10 nM IGF-1 for 1 week; (4) 200 nM Retinoic Acid for 2 weeks then 10 mM Nicotinamide +10 nM IGF-1 for 1 week; (5) 100 nM Retinoic Acid+30 nM Sonic Hedgehog for 2 weeks then 10 mM Nicotinamide+10 μM LY294002 for 1 week; (6) 2000 nM Retinoic Acid for 2 weeks then 10 mM Nicotinamide+10 μM LY294002 for 1 week; (7) 100 nM Retinoic Acid+30 nM Sonic Hedgehog for 2 weeks then 10 mM Nicotinamide+1 mM Sodium butyrate for 1 week; (8) 2000 nM Retinoic Acid for 2 weeks then 10 mM Nicotinamide+1 mM Sodium butyrate for 1 week.

FIG. 6. Insulin C-peptide yield from human NS cell cultures exposed to specific sequences of conditions and growth factors. Conditions: (1) 100 nM Retinoic Acid +30 nM Sonic Hedgehog for 2 weeks then 10 mM Nicotinamide+2 nM Activin A for 1 week; (2) 200 nM Retinoic Acid for 2 weeks then 10 mM Nicotinamide+2 nM Activin A for 1 week; (3) 100 nM Retinoic Acid+30 nM Sonic Hedgehog for 2 weeks then 10 mM Nicotinamide+10 nM IGF-1 for 1 week; (4) 200 nM Retinoic Acid for 2 weeks then 10 mM Nicotinamide+10 nM IGF-1 for 1 week; (5) 100 nM Retinoic Acid+30 nM Sonic Hedgehog for 2 weeks then 10 mM Nicotinamide+10 μM LY294002 for 1 week; (6) 200 nM Retinoic Acid for 2 weeks then 10 mM Nicotinamide+10 μM LY294002 for 1 week; (7) 100 nM Retinoic Acid+30 nM Sonic Hedgehog for 2 weeks then 10 mM Nicotinamide+1 mM Sodium butyrate for 1 week; (8) 2000nM Retinoic Acid for 2 weeks then 10 mM Nicotinamide+1 mM Sodium butyrate for 1 week.

FIG. 7. Proinsulin yield from human NS cell cultures exposed to specific sequences of conditions and growth factors. Conditions: (1) 100 nM Retinoic Acid+30 nM Sonic Hedgehog for 2 weeks then 10 mM Nicotinamide+2 nM Activin A for 1 week; (2) 200 nM Retinoic Acid for 2 weeks then 10 mM Nicotinamide+2 nM Activin A for 1 week; (3) 100 nM Retinoic Acid+30 nM Sonic Hedgehog for 2 weeks then 10 mM Nicotinamide+10 nM IGF-1 for 1 week; (4) 200 nM Retinoic Acid for 2 weeks then 10 mM Nicotinamide+10 nM IGF-1 for 1 week; (5) 100 nM Retinoic Acid+30 nM Sonic Hedgehog for 2 weeks then 10 mM Nicotinamide+10 μM LY294002 for 1 week; (6) 200 nM Retinoic Acid for 2 weeks then 10 mM Nicotinamide+10 μM LY294002 for 1 week; (7) 100 nM Retinoic Acid+30 nM Sonic Hedgehog for 2 weeks then 10 mM Nicotinamide+1 mM Sodium butyrate for 1 week; (8) 200 nM Retinoic Acid for 2 weeks then 10 mM Nicotinarnide+1 mM Sodium butyrate for 1 week.

FIG. 8: Cells were cultured for varying periods of time in the presence of retinoic acid, with and without sonic hedgehog.

FIG. 9: Semiquantitative RT-PCR analysis of human insulin mRNA from stage 1 and 2 NS-derived tissue (St. 1 and St. 2) and human islet control. Molecular weight standards (L) and GAPDH loading controls shown. Identity of the insulin and GAPDH products was confirmed by DNA sequencing.

FIG. 10: Insulin messenger RNA is expressed in stage 3 neurosphere-derived insulin-producing clusters. The negative control panel (sense; left panel) shows that there is little to no background staining of sectioned neurosphere clusters. Blue staining of the stage 3 cluster (middle panel) with the “anti-sense” human mRNA probe indicates that 40% of cells express insulin. Human pancreatic islets (right panel) are the positive control in this experiment.

FIG. 11: A. RT-PCR data demonstrating changes in gene expression during development of insulin-producing cell clusters from human neural stem cells. Lane 1 is undifferentiated neural stem cells, Lanes 2-4 correspond to stages 1, 2 and 3 respectively. Nestin, a marker of multipotent neural cells is expressed by neural stem cells in stages 1 and 2 but note extinguished expression of nestin by stage 3, in agreement with immunohistochemical data previously submitted. This is consistent with the notion that cells are differentiating during our procedure. Olig2 is also expressed in undifferentiated neural stem cells and its expression is also reduced. Thus, at least two markers reflect the observed loss of multipotency that is expected by treating cells with differentiating agents. En1 is a marker of spinal intemeurons and neurons and its expression in stages 2 and 3 is expected since retinoic acid treatment in stage 2 is known to induce differentiation of “caudal” neuronal cell types (like those found in spinal cord). Enl is also expressed in pancreatic islets so increased Enl expression may also reflect differentiation toward this cell type. HNF3-gamma, Cdx-1 and Ipf-1 are transcription factors known to be expressed in embryonic endoderm (the cell type from which islets emerge) in the foregut and midgut/hindgut. The increased expression of these markers provides good evidence for differentiation of neural stem cells toward an endoderm fate. Insulin expression by stage 3 (under the newer conditions used in our recently optimized protocol, which has lowered levels of glucose) is robust and requires addition of nicotinamide and IGF at stage 3. The absence of markers of mesoderm formation (brachyury, the vascular marker flk-1, β-globin and myosin light chain kinase 2, MLCK) supports the idea that little to no mesodermal differentiation occurs during differentiation of neural stem cells. Thus, in some ways, neural stem cells may provide advantages over embryonic stem cells, which have not yet been shown to differentiate endoderm without mesoderm. NCAM and GAPDH are used as loading controls for the gel electrQphoresis and show that an equivalent amount of sample was added to each RT-PCR mixture.

FIG. 11B RT-PCR data demonstrating changes in gene expression during development of insulin-producing cell clusters from human neural stem cells. Lane 1 is undifferentiated neural stem cells, Lanes 2-4 correspond to stages 1, 2 and 3 respectively. Lane 5 shows results from omission of reverse transcriptase (control) and lane 6 shows positive control expression (in human pancreas or liver). HNF3-α (FoxA3) and Pdx-1 are transcription factors known to be expressed in embryonic endoderm nproviding evidence for differentiation of neural stem cells toward an endoderm fate. Insulin mRNA is detected by stage 3.

FIG. 12: Raising the glucose level to 25 mM stimulates an approximately two-fold increase in the level of insulin released by IPCCs. Addition of 25 mM sucrose, which does not elicit insulin secretion by pancreatic islets, also does not elicit significant release of insulin by our IPCCs.

FIG. 13: Glucose responsiveness and cell fate in stage 3 NI grafts. (Top) release of human C-peptide after intraperitoneal glucose challenge 2 weeks after transplantation of 1000 NS-derived IPCCs. No circulating human C-peptide was detected prior to challenge (0 min.) but was readily detected 30 min. after challenge. (Middle) Lack of tumor formation 3 weeks after IPCC engraftment. Circle indicates graft site. (Bottom) Human C-peptide expression (brown stain) in sectioned IPCC graft recovered 3 weeks after transplantation. Nuclei counterstained blue.

### DETAILED DESCRIPTION

1. Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to refer to one or to more than one (i.e., to at least one) of the grammatical object of the article, unless context clearly indicates otherwise. By way of example, “an element” means one element or more than one element.

A “cell composition” is any composition of matter generated by human manipulation that comprises viable cells as a substantial component. A cell composition may comprise more than one type of viable cell. An “enriched cell composition” is a cell composition comprising a substantially greater purity (i.e. at least twice as pure) of a recognizable cell type than is found in any natural tissue. A “pure cell composition” is a cell composition that comprises at least about 75%, and optionally at least about 85%, 90% or 95% of a recognizable cell type. A recognizable cell type is generally one that has a reasonably uniform morphology, a characteristic set of two or more molecular markers and a functional characteristic. It is understood that there is likely to be some variation in certain characteristics even within a recognizable cell type. A cell composition may comprise, in addition to cells, essentially any component(s) that are compatible with the intended use for the cell composition. For example, a cell composition may include media, growth factors, pharmaceutically acceptable excipients, preservatives, a solid or semi-solid substrate, a porous matrix or scaffold, nonviable cells or a therapeutic agent.

The term “culturing” includes exposing cells to any condition. While “culturing” cells is often intended to promote growth of one or more cells, “culturing” cells need not promote or result in any cell growth, and the condition may even be lethal to a substantial portion of the cells.

A “cyclic AMP stimulating agent” or “cAMP stimulating agent” is any agent that causes an increase in cAMP mediated cell signaling. Exemplary cyclic AMP stimulating agents include forskolin and membrane diffusible cAMP analogues and phosphodiesterase inhibitors including 3-isobutly-1-methyl xanthine (IBMX).

A later cell is “derived” from an earlier cell if the later cell is descended from the earlier cell through one or more cell divisions. Where a cell culture is initiated with one or more initial cells, it may be inferred that cells growing up in the culture, even after one or more changes in culture condition, are derived from the initial cells. A later cell may still be considered derived from an earlier cell even if there has been an intervening genetic manipulation.

The term “including” is used herein to mean, and is used interchangeably with, the phrase “including but not limited to”.

An “islet cell differentiation factor” or “ICDF” is a factor that promotes the development of islet cell characteristics in a cell of pancreatic lineage. An ICDF may promote insulin production, maturation, storage or secretion in a cell that already produces insulin. Exemplary ICDFs include: IGF-1 AND IGF-1 AGONISTS, HGF, a cyclic AMP stimulating agent, exendin, GLP1, PPARγ ligand, sonic hedgehog, PACAP, growth hormone, P13K inhibitors and ADPRT inhibitors.

The term “marker” as used herein refers to a detectable aspect of a cell. For example, an insulin marker may include an insulin transcript or an insulin polypeptide, such as proinsulin, the alpha chain, the beta chain or the C peptide. A cell is “positive” for a marker if that marker is convincingly detected in the cell.

A “neural/endoderm caudalizing factor” refers to any factor, whether naturally occurring or artificial, that causes iumature cells of neural and/or endoderm derivation to adopt one or more characteristics of a caudal cell type, such as a spinal motor neuron or pancreatic cell. A neural/endoderm caudalizing factor is also intended to include mixtures of factors that collectively have a caudalizing effect on the appropriate cell types.

The term “nicotinamide agent” includes nicotinamide and analogs thereof that are biocompatible. Optionally, a nicotinamide agent has ADPRT inhibitory activity.

The term “or” is used herein to mean, and is used interchangeably with, the term “and/or”, unless context clearly indicates otherwise.

The term “pancreatic hormone” is used to refer to hormones produced by pancreatic islet cells, and particularly insulin, glucagon, pancreatic polypeptide and somatostatin.

The term “percent identical” refers to sequence identity between two amino acid sequences or between two nucleotide sequences. Percent identity can be determined by comparing a position in each sequence which may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MP SRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The term “P13K” refers to a phosphatidylinositol (PI) 3′-kinase, a family of proteins that phosphorylate the inositol ring of PI in the D-3 position. The canonical mammalian P13K is a heterodimeric complex that contains p85 and a 110-Kd protein (p110) (Carpenter et al. (1990) J. Biol. Chem. 265, 19704). The purified p85 subunit has a regulatory role while the 110-Kd subunit harbors the catalytic activity. Exemplary P13K inhibitors include wortmannin, LY294002, a P13K-targeted RNAi, etc.

A “poly-adenosine diphosphate ribosyl transferase inhibitor” or “ADPRT inhibitor” includes any compound or treatment that inhibits the ADPRT enzyme. Exemplary ADPRT inhibitors include nicotinamide and N-substituted benzamidines.

The term “stem cell” as used herein refers to an undifferentiated cell which is capable of proliferation and giving rise to at least one more differentiated cell type. “Totipotent stem cells” are stem cells that are capable of giving rise to any cell type of the organism from which the stem cells were obtained. “Pluripotent stem cells” are stem cells that are capable of giving rise to cells of the three major embryonic lineages, the endoderm, mesoderm and ectoderm. “Multipotent stem cells” are stem cells that are capable of giving rise to more than one type of more differentiated cell.

The term “stem cell” is also intended to include cells of varying developmental potential that may be obtained by somatic cell nuclear transfer or by causing a differentiated cell to undergo de-differentiation. For the purposes of this disclosure, a stem cell is named by the tissue from which it was obtained. For example, a “neural stem cell” is a stem cell obtained from a neural tissue (or a fluid, such as cerebrospinal fluid that is in contact with neural tissue), a “neuroendocrine stem cell” is a stem cell derived from a neuroendocrine tissue, such as the adrenal gland or the pituitary gland, but specifically excluding the pancreas. An “embryonic stem cell” is a stem cell obtained from an embryo. Many “tissues” are complex and actually contain several different stem cell types. For example, the skin may be considered a tissue, but skin contains neural stem cells of the peripheral nervous system, skin stem cells from the dermis, and stem cells from the blood circulating through the skin. Accordingly, in determining the classification of a stem cell, the true origin, including sub-tissue structures, should be carefully considered.

A “stem cell line” is an enriched or pure cell composition comprising a recognizably distinct stem cell type that, when cultured in appropriate conditions, self-propagates.

2. Methods for Generating Islet Cell Differentiation Factor-Responsive Cells and Pancreatic Hormone Producing Cells

In certain aspects, the disclosure relates to the discovery that neural and/or endoderm caudalizing factors, referred to collectively herein as “neural/endoderm caudalizing factors”, are useful in the process of producing insulin producing cells from stem cells, particularly neural, neuroendocrine and embryonic stem cells. In some instances, a neural/endoderm caudalizing factor renders a cell receptive to stimulation with an islet cell differentiation factor (“ICDF”). Accordingly, in certain aspects, the disclosure discloses methods for culturing stem cells to produce a population of ICDF-responsive cells, and in further embodiments, the disclosure 30 provides methods for using ICDF-responsive cells to produce insulin-producing cells and other cell type that produce distinctive pancreatic factors, such as glucagon, pancreatic polypeptide and somatostatin. In certain aspects, the disclosure discloses methods for obtaining ICDF-responsive cells and insulin-producing cells (as well as other pancreatic-type cells) from embryonic, neural or neuroendocrine stem cells, as well as the ICDF-responsive and insulin-producing cells themselves.

Certain embodiments of the methods disclosed herein are advantageous in part because they permit the generation of ICDF-responsive cells and insulin-producing cells from starting materials, such as neural or neuroendocrine stem cell lines, that are available, as a practical matter, in sufficient quantities for formation of a therapeutically effective insulin-producing implant. By contrast, for example, fetal pancreatic tissue, and particularly human fetal pancreatic tissue, is only available in small quantities, making it difficult or impossible to assemble sufficient material to form a therapeutically effective implant.

In terms of developmental biology, caudalizing factors are factors that cause cells to adopt the characteristics of more posterior (or “caudal”) cell types along the rostrocaudal axis, which roughly corresponds to an anterior-posterior head-tail axis. During development of the nervous system a precursor structure called the neural tube forms and cells along the rostrocaudal axis of the neural tube adopt different characteristics. Caudalizing factors cause, or participate in causing, cells of the neural tube to adopt caudal cell characteristics and develop into the cells of the posterior neural structures, such as the caudal hindbrain and spinal cord. Development of endoderm-derived tissues, such as the digestive tube, liver and pancreas, is also guided by caudalizing factors, and as described herein, pancreatic lineages may be generated by caudalization of endodermal cells.

This disclosure discloses, among other things, the novel finding that neural/endoderm caudalizing factors are useful for causing stem cells to develop the capacity to become pancreatic in nature. The term “neural/endoderm caudalizing factor” refers to any factor, whether naturally occurring or artificial, that causes immature cells of neural and/or endoderm derivation to adopt one or more characteristics of a caudal cell type, such as a spinal motor neuron or pancreatic cell. The caudalizing capability of a factor may be tested, for example, by culturing a neural explant such as a chick neural plate explant, in the presence of the factor and assessing the caudalizing effect on the explant cells. Stem cells in culture may also be exposed to the putative caudalizing factor and assessed for rostral or caudal character. Otx2 and En1 may be used as markers of rostral character in neural cells, while Hoxc5 and Hoxc6 may be used as indicators of caudal character. See, for example, Nordstrom et al. (2002) Nature Neurosci. 5:525-32; Wichterle et al. (2002) Cell 110(3):385-97.

Activators of retinoic acid signaling, including natural and artificial retinoids, are examples of neural/endoderm caudalizing factors. Three retinoic acid receptors (RARA, RARb, RARg, and their isoform) and three retinoid X receptors (RXRa, RXRb, RXRg, and their isoforms), are highly conserved across vertebrate species and are known to bind retinoids, particularly all-trans and 9-cis retinoic acid, and mediate transcriptional regulation. RARs and RXRs form hetero- and homodimer. Retinoic acid causes cells of the neural tube to adopt a more caudal fate. Accordingly, in certain embodiments, the disclosure provides methods for obtaining ICDF-responsive cells by culturing stem cells in the presence of an activator of retinoic acid signaling that has caudalizing activity (a “caudalizing retinoic acid signaling activator”). In certain preferred embodiments, the caudalizing retinoic acid signaling activator is all-trans or 9-cis retinoic acid, or a mixture thereof.

In certain embodiments, the caudalizing retinoic acid signaling activator is a retinoid having caudalizing activity (a “caudalizing retinoid”, which term includes all trans and 9-cis retinoic acid and other caudalizing retinoids). Examples of retinoids that may have caudalizing activity are described below and, in greater detail, in the published PCT disclosure WO03007950.

Retinoids are a class of compounds consisting of four isoprenoid units joined in a head to tail manner. Retinoids may be formally derived from a monocyclic parent compound containing five carbon-carbon double bonds and a functional group at the terminus of the acyclic portion. The basic retinoid structure is generally subdivided into three segments: the polar terminal end, the conjugated side chain, and the cyclohexenyl ring. The basic structures of the most common natural retinoids are called retinol, retinaldehyde, and retinoic acid. Examples include all-trans- (and cis)-retinyl ethers, all-trans- (and cis)-retinyl esters, all-trans- (and cis)-retinylamine retinylamine derivatives, all-trans- (and cis)-retinal derivatives, all-trans- (and cis)-retinoic acid esters), all-trans- (and cis)-retinoylamino acid, all-trans- (and cis)-retinamides. Retinoids thus, include side-chain modified cis and multi-cis retinoids such as, but not limited to, 13-cis-retinoic acid derivatives such as 13-cis-retinoic acid, N-ethyl-13-cis-retinamide, N-(2-hydroxyethyl)-13-cis-retinamide, N-(4-hydroxyphenyl) -13-cis-retinamide, N-(13-cis-retinoyl(leucine), and N-(13-cis-retinoyl)phenylalanine, bifunctional retinoic acid analogs such as 14-carboxyretinoic acid, ethyl 14-(ethoxycarbonyl)retinoate, and 14-[(ethylamino)carbonyl]-13-cis-retinoic acid. Retinoids also include ring-modified analogues such as the ring-modified all-trans-retinoic acid analogues including but not limited to alpha-retinoic acid, 4-hydroxyretinoic acid, phenyl analogue of retinoic acid, 4-methoxy-2,3,6-trimethylphenyl analogue of retinoic acid, 5,6-dihydroretinoic acid, 4-oxoretinoic acid, 3-pyridyl analogue of retinoic acid, dimethylacetylcyclopentenyl analogue of retinoic acid, 2-furyl analogue of retinoic acid, and the 3-thienyl analogue of retinoic acid. Ring-modified retinoids also include retinoid analogues in which the cyclohexenyl ring is replaced by napthoquinone-related structures.

Retinoids also include side-chain modified all-trans-retinoic acid analogues such as a C15 analogue of retinoic acid, a C17 analogue of retinoic acid, a C22 analogue of retinoic acid, an aryltriene analogue of retinoic acid, 7,8-dihydroretinoic acid, 8,10-dihydroretinoic acid, 11,12-dihydroretinoic acid. Other side chain modified retinoids include retinol, retinoic acid, and other retinoids with a partially or completely hydrogenated side chain. Still other retinoids having a modified side chain include, but are not limited to, retinol or retinoic acid derivatives in which selected double bonds of the side chain are replaced with amide, sulfonamide, or other groups such as, but not limited to, p-(5,6,7,8-tetrahydro-5,5,8,8-tetramethyl-2-hapht- alene-carboxamido)benzoic acid.

A large number of retinoids are commercially available (e.g., from Sigma Chemical Co., St. Louis, Mo. or from Aldrich Chemical Co., Inc., Milwaukee, Wis.). In addition, means of synthesizing and/or purifying retinoids are well known to those of skill in the art (see, e.g., Brahama et al. (1990) Meth. Enzymol., 189: 43-59; Klaus et al. (1990) Meth. Enzymol., 189: 3-14; Dawson et al. (1990) Meth. Enzymol., 189: 15-42; U.S. Pat. Nos. 5,648,091, 5,637,779, 5,639,919, 5,426,247, 4,876,400, and Kirk-Othmer, (1978) Encyclopedia of Chemical Technology, 24: 140).

In addition to retinoids, a non-retinoid activator of retinoic acid signaling having caudalizing activity may be used. Examples of non-retinoid activators of retinoic acid signaling may be found in the references provided below and in the literature generally. Accordingly, in certain embodiments, a caudalizing non-retinoid activators of retinoic acid signaling may be a chroman, thiochroman or 1,2,3,4-tetrahydroquinoline derivative as described in U.S. Pat. Nos. 4,980,369, 5,006,550, 5,015,658, 5,045,551, 5,089,509, 5,134,159, 5,162,546, 5,234,926, 5,248,777, 5,264,578, 5,272,156, 5,278,318, 5,324,744, 5,346,895, 5,346,915, 5,348,972, 5,348,975, 5,380,877, 5,399,561, 5,407,937. In addition, U.S. Pat. Nos. 4,740,519 (Shroot et al.), U.S. Pat. No. 4,826,969 (Maignan et al.) U.S. Pat. No. 4,326,055 (Loeliger et al.), U.S. Pat. No. 5,130,335 (Chandraratna et al.), U.S. Pat. No. 5,037,825 (Klaus et al.), U.S. Pat. No. 5,231,113 (Chandraratna et al.), U.S. Pat. No. 5,324,840 (Chandraratna), U.S. Pat. No. 5,344,959 (Chandraratna), U.S. Pat. No. 5,130,335 (Chandraratna et al.), Published European Patent Disclosure Nos. 0 176 034 A (Wuest et al.), 0 350 846 A (Klaus et al.), 0 176 032 A (Frickel et al.), 0 176 033 A (Frickel et al.), 0 253 302 A (Klaus et al.), 0 303 915 A (Bryce et al.), UK Patent Disclosure GB 2190378 A (Klaus et al.), German Patent Disclosure Nos. DE 3715955 A1 (Klaus et al.), DE 3602473 A1 (Wuest et al., and the articles J. Amer. Acad. Derm. 15: 756-764 (1986) (Spom et al.), Chem. Pharrn. Bull. 33: 404-407 (1985) (Shudo et al.), J. Med Chem. 31: 2182-2192 (1988) (Kagechika et al.), Chemistry and Biology of Synthetic Retinoids CRC Press Inc. 1990 pp.334-335, 354 (Dawson et al.), describe compounds with retinoid-like or related biological activity. U.S. Pat. No. 4,391,731 (Boller et al.) describes tetrahydronaphthalene derivatives that are useful in liquid crystal compositions.

An article by Kagechika et al. in J. Med. Chem 32:834 (1989) describes certain 6-(3-oxo-1-propenyl)-1,2,3,4-tetramethyl-1,2,3,4-tetrahydronaphthalene derivatives and related flavone compounds having retinoid-like activity. The articles by Shudo et al. in Chem. Pharm. Bull. 33:404 (1985) and by Jett et al. in Cancer Research 47:3523 (1987) describe or relate to further 3-oxo-1-propenyl derivatives (chalcone compounds) and their retinoid-like or related biological activity.

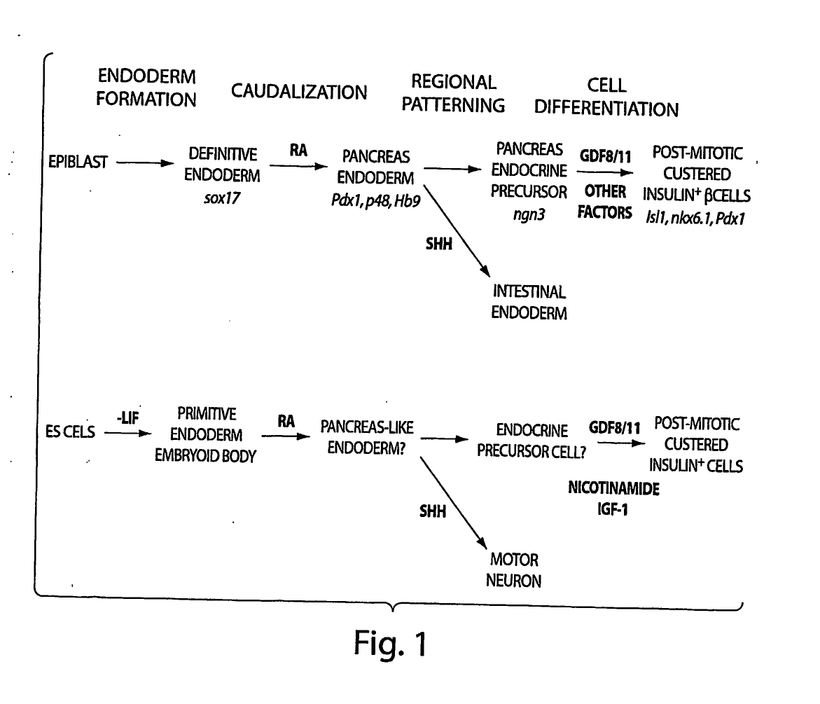

Many caudalizing factors are not structurally or functionally related to retinoids. For example, GDF-11 has endoderm caudalizing activity, as do certain other members of the TGF-beta family (however, as described herein, GDF-11 is preferably employed in causing caudalized cells to develop into insulin producing cells, see FIG. 1). Other caudalizing factors include Wnts and agonists of Wnt signaling and FGFs, such as FGF8. In addition, as shown herein, Sonic hedgehogs (SHH) antagonize the endoderm caudalizing effects of retinoic acid, and accordingly hedgehog antagonists, such as cyclopamine (and other veratrum alkaloids) and forskolin, may be employed as caudalizing factors. Methods described herein may employ caudalizing factors singly or in combination.

An exemplary method for generating ICDF-responsive cells comprises culturing cells of a human embryonic, neural or neuroendocrine stem cell line in a medium comprising growth factors such as leukemia inhibitory factor (LIF), epidermal growth factor (EGF) and basic fibroblast growth factor (bFGF). This is followed by culturing in a medium containing a neural/endoderm caudalizing factor such as a retinoid. Optionally, the second medium comprises insulin, transferrin and selenium. Optionally the second medium contains a steroid hormone such as progesterone. At least a portion of the resulting cells are ICDF-responsive cells.

ICDF-responsive cells are cells that respond to culturing with an islet cell differentiation factor by developing or strengthening one or more properties of a pancreatic islet cell type, such as an alpha cell, beta cell, delta cell or pancreatic polypeptide (PP) cell. Alpha, beta, delta and PP cells are, respectively, the endogenous pancreatic cell types responsible for production of glucagon, insulin, somatostatin and pancreatic polypeptide. Examples of properties of beta cells include: production of glucokinase, production of an insulin marker, such as an insulin transcript, proinsulin polypeptide, insulin alpha chain, insulin beta chain or C peptide and glucose-responsive production of insulin.

ICDFs are factors that are recognized as promoting the development of one or more properties of pancreatic islet cells in cells of pancreatic lineage and in ICDF-responsive cells. An ICDF may promote insulin production, maturation, storage or secretion in a cell that already produces insulin. Exemplary ICDFs include: IGF-1 AND IGF-1 agonists, hepatocyte growth factors (HGFs), a cyclic AMP stimulating agent, exendins, glucagon-like peptides (e.g. GLP1), PPARγ ligand, sonic hedgehog, PACAP, growth hormone, P13K inhibitors (e.g. LY294002, wortmannin), ADPRT inhibitors (e.g. benzamidine agents and certain nicotinamide agents, such as nicotinamide itself). Nicotinamide, IGF-1, IGF-1 agonists, GDF-11, GDF-8 and GDF-8/11 agonists are preferred ICDFs, that may be used in combination. As described herein, insulin-producing cells are often non-proliferative while their precursors are proliferative, and accordingly agents that inhibit proliferation may also be used as ICDFs, including agents such as rapamycin and cyclosporine A (P13K inhibitors may also have a growth inhibitor effect). LY294002 is 2-(4-Morpholinyl)-8-phenyl-4 H-1-benzopyran-4-one; as described by Vlahos, et al. (1994) J. Bidl., Chem., 269(7) 5241-5248, and is available from Calbiochem Corp., La Jolla Calif. Other inhibitors of P13K include wortmannin, viridin, viridiol, demethoxyviridin, and demethoxyviridiol (see, U.S. Pat. No. 5,276,167). Once viridin, viridiol, demethoxyviridin, and demethoxyviridiol, or other P13K inhibitors are isolated and purified, analogs of each may be prepared via well known methods to provide generally known compounds such as those illustrated by formula I of U.S. Pat. No. 5,276,167. The effect of P13K inhibitors may also be achieved by inhibiting a different target that is upstream or downstream of P13K signaling (i.e. P13K pathway inhibition). A novel or uncharacterized factor may be assessed for ICDF activity by contacting an ICDF-responsive cell, optionally prepared according to a method disclosed herein, with the test factor and detecting one or more islet cell markers, such as insulin.

In certain embodiments, a population of cells containing ICDF-responsive cells produces relatively low levels or undetectable levels of insulin, and optionally produces relatively low levels or undetectable levels of one or more additional pancreatic hormones. In preferred embodiments, the ICDF-responsive cells cultured with an islet cell differentiation factor produce at least three, four, five, seven or ten times as much insulin as the untreated ICDF-responsive cells. In preferred embodiments, a method disclosed herein provides apopulation of cells comprising ICDF-responsive cells and comprising at least 50% viable cells, and preferably at least 75% or at least 90% viable cells. in certain embodiments, ICDF-responsive cells are derived from neural, neuroendocrine or embryonic stem cells, and in such instances, a population of cells comprising ICDF-responsive cells may comprise cells retain one or more neural characteristics, such as beta tubulin III or nestin expression.

In certain embodiments, the disclosure provides insulin-producing cells derived from stem cells, and particularly embryonic, neural or neuroendocrine stem cells, and methods for preparing such cells. In certain embodiments, the disclosure provides insulin-producing cells prepared by culturing an ICDF-responsive cell in the presence of an ICDF. In certain aspects the disclosure provides methods for producing other pancreatic hormone producing cells, such as glucagon, somatostatin or PP producing cells, and cell compositions comprising a mixture of pancreatic hormone producing cell types.

An exemplary method for generating insulin-producing cells and other pancreatic hormone producing cells comprises culturing cells of a human neural or neuroendocrine stem cell line in a medium comprising growth factors such as leukemia inhibitory factor (LIF), epidermal growth factor (EGF) and basic fibroblast growth factor (bFGF). This is followed by culturing in a medium containing a neural/endoderm caudalizing factor such as a retinoid. Optionally, the second medium comprises insulin, transferrin and selenium. Optionally the second medium contains a steroid hormone such as progesterone. At least a portion of the resulting cells are ICDF-responsive cells. ICDF-responsive cells, and populations of cells comprising ICDF-responsive cells may be cultured in a third medium containing an ICDF, resulting in the development of insulin-producing cells. In a preferred embodiment, culturing with an ICDF includes culturing with nicotinamide, IGF-1 OR IGF-1 agonists, GDF-8, GDF-11, GDF-8/11 agonists, a P13K inhibitor or a combination thereof. Optionally, the third medium comprises insulin, transferrin and selenium. Optionally the third medium contains a steroid hormone such as progesterone. In certain embodiments, the second and third media are the same, but for the replacement of the caudalizing factor in the second medium with the ICDF in the third medium.

Insulin-producing cells may be produced in a variety of forms, including, preferably, insulin-producing cell clusters, but optionally in isolated cells, dispersed cell suspensions, confluent cell cultures or seeded on a matrix or other cell support. Other pancreatic hormone producing cells may also be produced in a variety of forms.

In further embodiments, the disclosure provides insulin-producing cell compositions in which at least about 50% of the cells are positive for insulin production, optionally at least 75% of the cells are positive for insulin production and preferably at least 85%, 90% or 95% of the cells are positive for insulin production. In certain embodiments, at least 75%, 85%, 90% or 95% of the cells have cytoplasmic insulin. Cytoplasmic insulin may be assessed, for example, by microscope in cells that have been stained with an anti-insulin antibody. In certain embodiments, most of the cells, and preferably greater than 80%, 90% or 95% of the cells, that produce insulin are negative for other pancreatic hormones that are not naturally produced by native pancreatic insulin-producing cells, such as glucagon. In certain embodiments, insulin-producing cells are produced in a cell composition comprising other cells that produce different pancreatic hormones. In certain embodiments, insulin-producing cells produce insulin at a level that is at least 0.5%, 1%, 2%, 3%, 5% or at least 10% of that estimated in native pancreatic beta cells. In certain embodiments, insulin-producing cells produce insulin at a level of at least 50 ng/mg total protein, and optionally at least 100, 200, 500, 750 or 1000 ng/mg total protein.

Optionally, insulin-producing cells and cell compositions are derived from neural stem cells, preferably neural stem cells of a neural or neuroendocrine stem cell line. In certain embodiments, insulin-producing cell compositions derived from neural stem cells comprise cells that retain one or more neural characteristics. Examples of neural characteristics include the expression of beta-tubulin m.

In certain embodiments, insulin-producing cell compositions comprise cells that are positive for one or more of the following markers: insulin (any of the various chains, including, for example, C-peptide, mature insulin or proinsulin), GLUT2, glucokinase, PDX-1, IAPP, SUR1, PC1/3, PC2 and KIR6.2. In certain embodiments, at least about 50%, 75% or 90% of the cells in an insulin-producing cell composition are not proliferative. Proliferating cells may be detected by a variety of ways known in the art, including staining with Ki67, a nuclear marker of proliferating cells, or incorporation of labeled nucleotide (e.g. tritiated thymidine or bromodeoxyuridine). In certain embodiments, at least about 50%, 75% or 90% of the cells in an insulin-producing cell composition are not apoptotic. Apoptosis may be measured, for example, by staining for TdT-mediated dUTP digoxigenin nick end labeling (also called “TUNEL” labeling). In certain embodiments, at least about 50%, 75% or 90% of the cells in an insulin-producing cell composition are viable.

In certain embodiments, the disclosure provides cells that produce a pancreatic hormone other than insulin, such as glucagon, somatostatin or pancreatic polypeptide, and such cells may occur in cell compositions with each other and with insulin-producing cells. In certain embodiments, at least 50%, and preferably at least 75%, 85% or 90%, of cells that produce a pancreatic hormone selected from the group consisting of: insulin, glucagon, somatostatin and pancreatic polypeptide do not produce any of the other three members of the group. In other words, in certain preferred embodiments, cells tend to mimic the phenotypes of alpha, beta, gamma and PP-producing cells of a normal pancreas. Certain methods disclosed herein result in the production of islet like cell clusters that comprise cells of each of the following types: insulin-producing, glucagon-producing somatostatin-producing and pancreatic polypeptide producing. Optionally fewer than 50%, and preferably fewer than 25%, 15% and 10% of cells in an islet like cell cluster are apoptotic. Optionally fewer than 50%, and preferably fewer than 25%, 15% and 10% of cells in an islet like cell cluster are apoptotic or proliferative.

Stem cells for use in the methods disclosed herein may be essentially any stem cell that has not lost the potential to become a pancreatic hormone-producing cell. The term “stem cell” as used herein refers to an undifferentiated cell which is capable of proliferation and giving rise to at least one more differentiated cell type. Stem cells may be totipotent, pluripotent stem cells or multipotent. Stem cells may also be obtained by somatic cell nuclear transfer or by causing a differentiated cell to undergo de-differentiation. In certain embodiments, stem cells for use with the disclosed methods may be impure, such as stem cells nested in a tissue or in a suspension obtained from a tissue sample. It is now widely believed that most adult tissues include small populations of stem cells, as that term is used herein. Stem cells may also be enriched from tissue samples, and may optionally be purified stem cells. Stem cells may also be used from stem cell lines, and preferably from well-characterized and established stem cell lines. Tissue may be embryonic or “adult” as the term is used herein, including fetal, infant, child and mature animal tissue. Cells need not be obtained from a tissue, and other cell-containing sources that are not generally considered “tissues” may be employed (e.g. cerebrospinal fluid and mucus or secreted fluids of the lung or gut). In preferred embodiments, where cells are to be used for therapy in a human, the stem cells are human stem cells.

In certain embodiments, a stem cell for use in disclosed methods is a stem cell of neural or neuroendocrine origin, such as a stem cell from the central nervous system (see, for example U.S. Pat. Nos. 6,468,794; 6,040,180; 5,753,506; 5,766,948), neural crest (see, for example, U.S. Pat. Nos. 5,589,376; 5,824,489), the olfactory bulb or peripheral neural tissues (see, for example, Published US Patent Disclosures 20030003574; 20020123143; 20020016002 and Gritti et al. 2002 J Neurosci 22(2):437-45), the spinal cord (see, for example, U.S. Pat. Nos. 6,361,996, 5,851,832) or a neuroendocrine lineage, such as the adrenal gland, pituitary gland or certain portions of the gut (see, for example, U.S. Pat. Nos. 6,171,610 and PC12 cells as described in Kimura et al. 1994 J. Biol. Chem. 269: 18961-67). In preferred embodiments, a neural stem cell is obtained from a peripheral tissue or an easily healed tissue from a patient in need of cells that produce a pancreatic hormone, thereby providing an autologous population of cells for transplant. In another preferred embodiment, a neural stem cell for use in method disclosed herein is selected from a cell population containing neural or neural-derived cells for cells by binding to a monoclonal antibody AC133 or to a monoclonal antibody 5E12, as described in U.S. Pat. No. 6,468,794. Cells of this type are deposited with the ATCC, 10801 University Blvd., Manassas, Va. 20110-2209, under ATCC accession numbers PTA-993 and PTA-994.

In certain embodiments, a stem cell for use in the methods disclosed herein is an embryonic stem cell, such as a cell of an embryonic stem cell line. Stem cell lines are preferably derived from mammals, such as rodents (e.g. mouse or rat), primates (e.g. monkeys, chimpanzees or humans), pigs, and ruminants (e.g. cows, sheep and goats). Examples of mouse embryonic stem cells include: the JM1 ES cell line described in M. Qiu et al., Genes Dev 9, 2523 (1995), and the ROSA line described in G. Friedrich, P. Soriano, Genes Dev 5, 1513 (1991), and mouse ES cells described in U.S. Pat. No. 6,190,910. Many other mouse ES lines are available from Jackson Laboratories (Bar Harbor, Me.). Examples of human embryonic stem cells include those available through the following suppliers: Arcos Bioscience, Inc., Foster City, Calif., CyThera, Inc., San Diego, Calif., BresaGen, Inc., Athens, Ga., ES Cell International, Melbourne, Australia, Geron Corporation, Menlo Park, Calif., Göteborg University, Göteborg, Sweden, Karolinska Institute, Stockholm, Sweden, Maria Biotech Co. Ltd.—Maria Infertility Hospital Medical Institute, Seoul, Korea, MizMedi Hospital—Seoul National University, Seoul, Korea, National Centre for Biological Sciences/Tata Institute of Fundamental Research, Bangalore, India, Pochon CHA University, Seoul, Korea, Reliance Life Sciences, Mumbai, India, Technion University, Haifa, Israel, University of California, San Francisco, Calif., and Wisconsin Alumni Research Foundation, Madison, Wis. In addition, examples of embryonic stem cells are described in the following U.S. patents and published patent applications: 6,245,566; 6,200,806; 6,090,622; 6,331,406; 6,090,622; 5,843,780; 20020045259; 20020068045. In preferred embodiments, the human ES cells are selected from the list of approved cell lines provided by the National Institutes of Health and accessible at http://escr.nih.gov. In certain preferred embodiments, a stem cell line is selected from the group consisting of: the WA09 line obtained from Dr. J. Thomson (Univ. of Wisconsin) and the UC01 and UC06 lines, both on the current NIH registry. A stem cell line, as the term is used herein, may include cells cultured directly from a tissue sample in such a way as to enrich for one or more types of stem cells. A passaged stem cell line is one that has been propagated through at least two media changes or growth substrate changes since being obtained from a tissue sample.

In certain embodiments, hematopoietic or mesenchymal stem cells may be employed in a disclosed method. Recent studies suggest that marrow-derived bematopoietic (HSCs) and mesenchymal stem cells (MSCs), which are readily isolated, have a broader differentiation potential than previously recognized. Purified HSCs not only give rise to all cells in blood, but can also develop into cells normally derived from endoderm, like hepatocytes (Krause et al., 2001, Cell 105: 369-77; Lagasse et al., 2000 Nat Med 6: 1229-34). MSCs appear to be similarly multipotent, producing progeny that can, for example, express neural cell markers (Pittenger et al., 1999 Science 284: 143-7; Zhao et al., 2002 Exp Neurol 174: 11-20). Examples of hematopoietic stem cells include those described in U.S. Pat. Nos. 4,714,680; 5,061,620; 5,437,994; 5,914,108; 5,925,567; 5,763,197; 5,750,397; 5,716,827; 5,643,741; 5,061,620. Examples of mesenchymal stem cells include those described in U.S. Pat. Nos. 5,486,359; 5,827,735; 5,942,225; 5,972,703, those described in PCT publication nos. WO00/53795; WO00/02654; WO 98/20907, and those described in Pittenger et al. and Zhao et al., supra.

Stem cell lines are preferably derived from mammals, such as rodents (e.g. mouse or rat), primates (e.g. monkeys, chimpanzees or humans), pigs, and ruminants (e.g. cows, sheep and goats), and particularly from humans. In certain embodiments, stem cells are derived from an autologous source or an HLA-type matched source. For example, stem cells may be obtained from a subject in need of pancreatic hormone-producing cells (e.g. diabetic patients in need of insulin-producing cells) and cultured by a method described herein to generate autologous insulin-producing cells. Other sources of stem cells are easily obtained from a subject, such as stem cells from muscle tissue, stem cells from skin (dermis or epidermis) and stem cells from fat. Insulin-producing cells may also be derived from banked stem cell sources, such as banked amniotic epithelial stem cells or banked umbilical cord blood cells.

In some instances, it may be desirable to obtain adult stem cells, such as neural or neuroendocrine stem cells for use in generating insulin producing cells to administer to a patient. Such cells may be obtained directly from the patient. Such cells may also be obtained from another individual, preferably an individual whose cells will have a reduced risk of rejection after administration to the subject. Donors with cells at reduced risk of rejection include, for example, close family members and HLA-matched donors. Tissues containing one or more cells of the central or eripheral nervous systems may be used, as well as tissues containing one or more cells of a neuroendocrine tissue (note that as used herein, the term neuroendocrine is intended to explicitly exclude pancreatic cells). Multipotent neural stem cells, unlike embryonic stem cells, may be derived from post-natal animals by trans-cranial, olfactory bulb, or spinal cord biopsy (Roisen et al Brain Res. 2001 Jan 26;890(1): 11-22; U.S. Patent Application Publication Nos. 20030003574 “Multipotent stem cells from peripheral tissues and uses thereof”, 20020123143 “Multipotent stem cells from peripheral tissues and uses thereof” and 20020016002 “Multipotent neural stem cells from peripheral tissues and uses thereof”. Tissues that may contain CNS or PNS derived neural stem cells include skin, spinal cord, cranial tissue, olfactory bulb, muscle (including neuromuscular junctions), bone and essentially any innervated structure.

In certain embodiments, a stem cell may be derived from a cell fusion or dedifferentiation process, such as described in the following US patent disclosure: 20020090722, and in the following PCT disclosures: WO200238741, WO0151611, WO9963061, WO9607732.

In some preferred embodiments, a stem cell line should be compliant with good tissue practice guidelines set for the by the U.S. Food and Drug Administration (FDA) or equivalent regulatory agency in another country. Methods to develop such a cell line may include donor testing, and avoidance of exposure to non-human cells and products during derivation of the stem cell lines. Preferably the stem cell line can be prepared and used without the use of a feeder layer or any type of virus or viral vector.

In certain preferred embodiments, both the stem cells and differentiated cells of the methods and compositions disclosed herein have a wild-type genotype, meaning that the genotype of the cells is a genotype that may be found in a subject organism naturally. For example, cells having chromosomal rearragements as a result of culture treatments are not cells having a wild-type genotype. As a further example, cells that have been transfected with an integrating nucleic acid construct will not (except in cases of perfect excision) have a wild-type genotype. The term “genotype” does not refer to peripheral modifications to the genomic nucleic acids, such as methylation, and therefore, cells having a naturally occurring genetic makeup may have unnatural phenotypes as an effect of changes in methylation or other modifications.

Any of the various factors and reagents described herein, including caudalizing factors and ICDFs, may be replaced or used in combination with functional analogs. A functional analog is a structurally similar molecule having at least 10%, and preferably at least 50%, of the activity of the factor or reagent. In the case of polypeptide factors, such as IGF-1, GDF-11 and GDF-8, a functional analog may be simply a version using one or more modified amino acids but retaining the same sequence, or a functional analog may be a polypeptide having at least 80% amino acid sequence identity to the polypeptide factor, and preferably at least 90% or 95% sequence identity. Functional analogs may be identified from combinatorial libraries by the use of high-throughput screens. A combinatorial chemical library is a collection of diverse chemical compounds. Such libraries may be generated by chemical synthesis or biological synthesis by combining a number of simpler chemical subunits. For example, a linear combinatorial chemical library such as a polypeptide library is formed by combining a set of amino acids in as many ways as possible for a given polypeptide length. The functionality of a candidate functional analog may be evaluated by using a published assay for the activity of the agent to be replaced. Millions of chemical compounds can be synthesized through such combinatorial mixing of subunits. Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J Pept. Prot. Res., 37: 487-493, Houghton et al. (1991) Nature, 354: 84-88). Peptide synthesis is by no means the only approach envisioned and intended for use with the present disclosure. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random bio-oligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J Amer. Chem. Soc. 114: 6568), nonpeptidal peptidomimetics with a .beta.-D-Glucose scaffolding (Hirschmann et al., (1992) J Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J Org. Chem. 59: 658). See, generally, Gordon et al., (1994) J Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

3. Administration of Insulin-Producing Cells and Cells Producing Other Pancreatic Hormones

In certain aspects, the disclosure relates to methods for ameliorating, in a subject, a condition related to insufficient pancreatic function by administering to the subject an effective amount of insulin-producing cells or cells producing other pancreatic hormones or a mixture thereof, as needed. In the case of a subject in need of insulin, preferably a sufficient amount of cells are administered to a subject to cause an increase in blood insulin levels or an improvement in glucose homeostasis. Glucose homeostasis may be tested by administering a dose of glucose and monitoring the kinetics with which blood glucose levels decline. Conditions related to insufficient pancreatic fuiction include the various forms of diabetes mellitus (e.g. type I and type II), NOD mice (a type I diabetes model), the streptozotocin-induced diabetes rodent model, surgically-induced diabetes models and diseases resulting from dysfunctional islet growth (e.g. insulinomas). Administration of insulin-producing cells may not produce a permanent ameliorating effect, and periodic dosing, such as on a weekly, monthly or yearly basis may be beneficial. in preferred embodiments, an effective dose of insulin-producing cells comprises administering at least a number of cells that is equivalent to the number of islets that is naturally present in the subject organism. For example, mice have about 300-500 islets, rats have about 3000-5000 islets and humans have about 1,000,000 islets, and accordingly, a preferred dosage is cells equivalent to about 300-500 islets for a mouse, about 3000-5000 islets for a rat and about 1,000,000 islets for a human. The number of islets per organism is proportional to average body mass (20-30 grams, mouse, 200-300 grams, rat, 60-70 kilograms, human) and it may be desirable to administer a dosage that is proportional to body mass of the subject. In instances when the cells to be implanted are less efficient at producing insulin than a native islet, or where insulin-producing cells are subject to cell mortality (e.g. in the case of host immune system-mediated rejection), the dosage may be increased proportionally. In certain instances, it may be impractical to deliver a full islet-equivalent of cells, and therefore doses that are equivalent to about one-half, one-quarter, one-tenth, one-twentieth or fewer of the islets naturally present in the organisms may also be used.

In certain embodiments, the disclosure relates to therapeutic compositions comprising insulin-producing cells or cells producing other pancreatic hormones, and methods for making such therapeutic compositions. Therapeutic compositions include an insulin-producing cell composition disclosed herein or an insulin-producing cell composition made by the methods disclosed herein, as well as mixtures comprising such insulin-producing cell compositions and a therapeutic excipient. Examples of therapeutic excipients include matrices, scaffolds or other substrates to which cells may attach (optionally formed as solid or hollow beads, tubes, or membranes), as well as reagents that are useful in facilitating administration (e.g. buffers and salts), preserving the cells (e.g. chelators such as sorbates, EDTA, EGTA, or quaternary amines or other antibiotics), or promoting engraftment.

Cells may be encapsulated in a membrane to avoid immune rejection. By manipulation of the membrane permeability, so as to allow free diffusion of glucose and insulin back and forth through the membrane, yet block passage of antibodies and lymphocytes, normoglycemia may be maintained (Sullivan et al. (1991) Science 252:718). In a second approach, hollow fibers containing cells may be immobilized in a polysaccharide alginate. (Lacey et al. (1991) Science 254:1782). Cells may be placed in microcapsules composed of alginate or polyacrylates. (Lim et al. (1980) Science 210:908; O'Shea et al. (1984) Biochim. Biochys. Acta. 840:133; Sugamori et al. (1989) Trans. Am. Soc. Artif. Intern. Organs 35:791; Levesque et al. (1992) Endocrinology 130:644; and Lim et al. (1992) Transplantation 53:1180).

Additional methods for encapsulating cells are known in the art. (Aebischer et al. U.S. Pat. No. 4,892,538; Aebischer et al. U.S. Pat. No. 5,106,627; Hoffrnan et al. (1990) Expt. Neurobiol. 110:39-44; Jaeger et al. (1990) Prog. Brain Res. 82:41-46; and Aebischer et al. (1991) J. Biomech. Eng. 113:178-183, U.S. Pat. No. 4,391,909; U.S. Pat. No. 4,353,888; Sugamori et al. (1989) Trans. Am. Artif. Intern. Organs 35:791-799; Sefton et al. (1987) Biotehnol. Bioeng. 29:1135-1143; and Aebischer et al. (1991) Biomaterials 12:50-55).

The site of implantation of insulin-producing cell compositions may be selected by one of skill in the art. In general, such as site preferably has adequate blood perfusion to allow the cells to sense blood conditions and secrete hormones and other factors into the general circulation. Exemplary implantation sites include the liver (via portal vein injection), the peritoneal cavity, the kidney capsule and the pancreas.

Cells described herein may be implanted in a non-human animal, especially a primate or a rodent, and accordingly, in further embodiments, the disclosure provides non-human animals that comprise an insulin-producing cell composition as disclosed herein. Such animals may be useful, for example, for screening compounds that may affect graft performance in vivo.

4. Methods for Assessing Candidate Islet Cell Differentiation Factors and Other Test Compounds

In certain embodiments, the disclosure relates to methods employing the ICDF-responsive cells and insulin-producing cells of the disclosure.

In certain aspects the disclosure provides methods for assessing whether a test agent has islet cell differentiation factor activity. An exemplary embodiment of such a method may comprise contacting cells that are receptive to treatment with an islet cell differentiation factor and detecting an islet cell marker. Generally, a test agent that stimulates the formation of cells expressing islet cell markers has ICDF activity. The term “islet cell marker” is intended to include any phenotype that is distinctive of one or more islet cell types, including various protein, nucleic acid, morphological, biochemical (e.g. metabolic or transport) or other phenotypes. Examples of islet cell markers include the following polypeptides or the corresponding RNA transcript: insulin (any of the various chains, including, for example, C-peptide, mature insulin or proinsulin), GLUT2, glucokinase, PDX-1, IAPP, SUR1, PC1/3, PC2, KIR6.2, pancreatic polypeptide, somatostatin, glucagon, glucokinase and C-peptide. In an illustrative embodiment, the subject cells can be used to screen various compounds or natural products, such as small molecules or growth factors. The efficacy of the test agent can be assessed by generating dose response curves. A control assay can also be performed to provide a baseline for comparison.

In certain embodiments, methods of the disclosure relate to the identification of pancreatic developmental markers. For example, expression patterns of established markers of endoderm and islet development may be monitored at one or more stages of differentiation of stem cells into ICDF-responsive and insulin-producing cells. Markers may be assessed using standard methods, including Northern blotting, RT-PCR, in situ hybridization (ISH), immunohistochemistry (IHC) as well as nucleic acid or protein array or microarray-based methods. In certain embodiments, monitoring production of one or more gene products will be useful to identify candidate cell-surface proteins for FACS-based purification strategies for insulin-producing cell precursors.

In certain embodiments, the disclosure provides methods for identifying affinity reagent that bind to cells at various stages of pancreatic development. Affinity reagents include antibodies, and preferably monoclonal antibodies, targeting peptides (e.g. peptides selected from a high diversity phage display library), RNA or DNA aptamers. The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody includes polyclonal, monoclonal, or other purified preparations of antibodies and recombinant antibodies. In certain embodiments, ICDF-responsive or pancreatic hormone-producing cells, particularly insulin producing cells, may be used to screen a plurality of affinity reagents. The cells themselves may be used for the screening, or membrane or protein extracts may be used. Likewise, cell surface proteins may be selectively labeled and used to screen a plurality of affinity reagents. In a preferred embodiment, the plurality of affinity reagents to be screened is a library of monoclonal antibodies. An affinity reagent detected as binding to a cell such as an ICDF-responsive or pancreatic hormone-producing cell may be tested on tissue samples for capability to detect particular subpopulations of pancreatic or pre-pancreatic cells, and it is of particular interest to identify affinity reagents that are useful in the identification of populations of cells that are precursors of beta cells or other islet cells.

Yet another aspect of the present disclosure provides methods for screening various compounds for their ability to modulate insulin-producing cells, such as, for example, by affecting growth, proliferation, maturation or differentiation, or by affecting insulin production, secretion or storage, as well as compounds that may mprove graft performance (e.g. result in a longer-lasting graft, improved insulin roduction, or changes in proteins that interact with the host immune system). In an illustrative embodiment, the subject cells can be used to screen various compounds or natural products, such as small molecules or growth factors. Such compounds may be tested for essentially any effect, with exemplary effects being cell proliferation or differentiation, insulin production, or cell death. In further embodiments, insulin-producing cells may used to test the activity of compounds/factors to promote survival and maturation, and further, since certain cells produced according to methods disclosed herein have one or more properties of islet cells, specifically β-cells, such cells may be used to identify factors (or genes) that regulate production, processing, storage, secretion, and degradation of insulin or other relevant proteins (like IAPP, glucagon, including pro-glucagon, GLPs, etc) produced in pancreatic islets. In further embodiments, an insulin-producing cell may be modified, such as by genetic modification, to become hyperproliferative. Such hyperproliferative cells may be contacted with compounds to identify, for example, anti-proliferative and anti-neoplastic agents (e.g. agents that inhibit cell growth or promote cell death). The efficacy of the compound can be assessed by generating dose response curves from data obtained using various concentrations of the compound. A control assay can also be performed to provide a baseline for comparison. Identification of the progenitor cell population(s) amplified in response to a given test agent can be carried out according to such phenotyping as described above. Assays such as those described above may be carried out in vitro (e.g. with cells in culture) or in vivo (e.g. with cell implanted in a subject).

5. Methods for Identifving Stem Cells

In certain embodiments, the disclosure relates to methods for identifying a cell that has the potential to develop into a pancreatic cell, and particularly an insulin-producing cell. In one aspect, the method comprises providing a stem cell line, or other multipotent cell line, and differentiating the cell line so as to obtain an insulin-producing cell composition. At the beginning of the differentiation process, or at some stage within the differentiation process, the differentiating cells are mixed with a cell of interest. The differentiation of the cell of interest may then be assessed. A cell of interest that is able to differentiate into an insulin-producing cell is a cell that has the potential to develop into an insulin-producing cell. In further embodiments, the cell may be assessed for the production of other pancreatic products, such as glucagons, to identify cells that have the potential to develop into other types of pancreatic cells. In certain embodiments, a pancreatic tissue (e.g. ductal tissue, adult pancreatic tissue, fetal pancreatic tissue, etc.) may be dissociated into a cell suspension, and clumps of cells or single cells are used as the cell of interest in the above method embodiments, thereby permitting a rapid screen of pancreatic cells for candidate pancreatic progenitors.

In one embodiment, insulin-producing cell compositions and methods for generating such compositions may be used to assess the developmental potential of a cell of interest. In some embodiments, the developmental potential of a cell of interest may be determined by mixing the cell of interest with cells during the process of making ICDF-responsive or insulin-producing cells (i.e. co-culturing).

The cell of interest is then tracked (for example by a transgenic marker) to determine the types of cells that arise from it. In an exemplary embodiment, the cell of interest is mixed with differentiating neural or neuroendocrine stem cells.

In certain embodiments, culture systems for making insulin-producing cell compositions may be used as part of an assay to identify candidate pancreatic endocrine precursor cells. Current evidence suggest that such precursors exist as single cells or small cell clusters within or closely associated with pancreatic epithelium. In certain embodiments, cell compositions in the process of differentiating into ICDF-responsive or insulin-producing cells provide the appropriate cellular microenvironment to permit pancreas-derived endoderm to integrate and differentiate. In certain embodiments, cells of a pancreatic tissue are fractionated and mixed, either as populations of cells or as single cells, into cells being differentiated into insulin-producing cell compositions. Cells of the pancreatic tissue that develop into insulin-producing cells are candidate pancreatic stem cells.

In certain embodiments, instead of a co-culture, a fraction of cells that are in the process of differentiating into insulin-producing cell compositions may be used in the culture medium of the cells of interest. Fractions that may be used include conditioned media or other preparations of secreted material, extracellular matrix, membrane preparations, total soluble protein, soluble cellular protein and other portions of cells that are in the process of differentiating into ICDF-responsive or insulin-producing cells.

The disclosure may be more readily understood by reference to the following examples, which are included merely for purposes of illustration of certain aspects and embodiments of the present disclosure, and are not intended to limit the disclosure.

### EXAMPLES

1. Isulin-Producing Cells Derived from Neural Stem Cells

The following example demonstrates the production of insulin-producing cells from neural stem cells. The level of insulin measured in the human NS cell-derived clusters is at least 0.5-3% of levels believed to be contained in pancreatic islets of Langerhans, the sole source of insulin in humans after birth. Evidence of de novo insulin synthesis in these cells is provided by detection of the proinsulin-derived cleavage product, C-peptide, and by co-expression of several known pancreatic β-cell markers in these insulin-producing cell clusters (FIGS. 1-3).

The results flurther demonstrate that sequential additions of retinoic acid and nicotinamide with either insulin-like growth factors, or with the a phosphatidylinositol-3-kinase inhibitor such as compound LY294002, has a significant effect in enhancing insulin production and cellular maturation of NS cell-derived insulin-producing cell clusters.

Cells:

Human NS cells from StemCells Inc. (Palo Alto, Calif.). Until stage 1, cells are maintained in neurospheres as described in Uchida et al, 2000, Proc Natl Acad Sci U S A. 2000 Dec. 19;97(26):14720-5.

Cell Culture Medium

Cell culture medium should be prepared using aseptic technique.

Once prepared the solutions should be stored in the refrigerator at 4° C. on the shelf for up to 4 week.

Incubators, Refrigerators, and Freezers

The tissue culture are kept at 37° C. and 5% CO₂.

Reagents

Bovine basic Fibroblast Growth Factor (bFGF) (20 ng/ml) [R&D Systems #133-FB-025]
30% Bovine serum albumin [Sigma A9576]
Collagenase H (0.5 mg/ml) [Boerhringer Mannheim #1087789]
Dimethyl Sulfoxide [Fisher #BP231-1]
DMEM/F-12 [GIBCO-BRL #10565-018]
0.02% EDTA/PBS solution [Sigma 011K2309]
Fibronectin [Sigma #F-4759]
D(+)-Glucose [Sigma #G-5146]
L-Glutamine [G-8540]
Heparin (0.2 mg/ml 100×) [Sigma #H-3149]
Human Epidermal Growth Factor (EGF) (20 μg/ml)[R&D Systems #236-EG-200]
Hydrochloric acid (HCl) (1N)
Insulin 5 mg/ml [Sigma #I-6634]
Insulin-like Growth Factor-1 (IGF-1) [R&D Systems #291-G1]
Leukemia Inhibitory Factor (LIF) ([Chemicon #LIF1010]
Ly294002 (10 μM) [Calbiochem #440202]
N2 Supplement 100×[Invitrogen #17502]
Nicotinamide (10 mM) [Sigma #N0636]
Penicillin-Streptomycin (Pen/Strep) 100×[Gibco-BRL #15140-122]
Phosphate-Buffered Saline (PBS) [Gibco-BRL #14190-250]
Poly -L-Ornithine [Sigma #P-4957]
Progesterone 2 mM stock (PG) [Sigma #P-8783]
Putrescine 1 M stock (Ptr) [Sigma #P-5780]
Sodium Bicarbonate [Sigma #S-5761]
Sodium Selenite (300 μM) [Sigma #S-5261]
all-trans-Retinoic acid [Sigma R2625]
X-VIVO 15 [BioWhittaker #04-418Q]
Cell Strainer (70 μm) [Fisher #08-7712]
6 well Cell Culture Plates [Fisher # 152795]
75 cm² Cell Culture Flask [Fisher # 07-200-68]
   Media
   Stage 1: X-VIVO media
500 ml X-VIVO media
5 ml N2 supplement
5 ml Heparin (2 μg/ml)
0.5 ml LIF (final: 1 μg/ml)
0.5 ml human EGF (final: 20 ng/ml)
10 ml bFGF (final: 20 ng/ml)
   Stage 2 and 3: N₂ Media
250 ml DMEM/F12 media
50 mg Apo-transferrin
775 mg D(+)-Glucose
36.5 mg L-Glutamine
845 mg Sodium bicarbonate
Adjust the pH to 7.1 to 7.2 with cell culture grade IN HCI.
Adjust the volume to 500 ml with pure ddH₂O.
Filter through a 0.22 μm filter.
   For 500 ml N₂ Media, Now Add the Following:
2.5 ml insulin (final: 25 μg/ml)
100 μl Progesterone (final: 20 nM)
50 μl Putrescine (1M) (final: 100 μM)
50 μl Sodium selenite (final: 30 nM)
5 ml Pen/Strep (×1)
   Stage 2
X ml N₂ media
2 μM all-trans-Retinoic acid
   Stage 3
X ml N₂ media
10 mM Nicotinamide Growth factors (10 μM Ly294002 or 10 nM IGF-1)
   Procedure
   Stage 1: Day 1 (1)
1. Thaw one vial of hNS cells (2×10⁶ cells/vial) in the 37° C. water bath for two minutes.
2. Gently add the cell suspension to Stage 1 X-VIVO media and centrifuge for 5 minutes at 1000 rpm to pellet the cells.
3. While the cells are spinning, add 8 ml of X-VIVO media to 75 cm² cell culture flask.
4. Once the cells are done spinning, aspirate of the supernatant and add 2 ml of X-VIVO media and resuspend hNS cells.
5. Add 2 ml of cell suspension to 75 cm² cell culture flask and place in the incubator.
   Stage 1: Day 7 (7)
1. Add 10 ml of fresh X-VIVO media to culture flask.
   Stage 1: Day 13 (13)
1. Coat plates with 45 μg/ml poly-L-ornithine
2. Add 2 ml of poly-L-ornithine solution to each well of a 6-well plate.
3. Place in 37° C. incubator overnight.
   Stage 2: Day 1 (14)
1. Aspirate off poly-L-ornithine solution.
2. Add 2 ml of PBS to each well of a 6-well plate.
3. Incubate at 37° C. incubator for 1 hour.
4. Aspirate off PBS and add 3 μg/ml fibronectin solution.
5. Incubate at 37° C. incubator for at least 1 hour.
6. Make Stage 2 media.
7. Harvest the neurospheres into a 50 ml Falcon tube.
8. Let the neurospheres settle for 10 minutes.
9. Aspirate off the solution and add Stage 2 media.
10. Plate on the pre-coated plates. (plate the NS cells in 1×75 cm² cell culture flask on 4×6-well plates)
11. Change media every other day for 2 weeks.
   Stage 3: Day 1 (28)
1. Change to Stage 3 media including growth factors.
2. Change media every other day for 1 week.
3. Harvest cells for analysis or experiment.
   Related References: Hori et al, 2002 Proc Natl Acad Sci USA 99: 16105-110.
Stafford D, Prince VE, 2002 Curr Biol. 12:1215-20. Wichterle et al. 2002 Cell 110:385-97.
   2. Retinoic Acid and Sonic Hedgehog have Opposing Effects on Development of Insulin-Producing Cells.

Neural stem cells from StemCells Inc. (Palo Alto, Calif.) were cultured as described above, except that various combinations of caudalizing factors were assessed along with SHH.

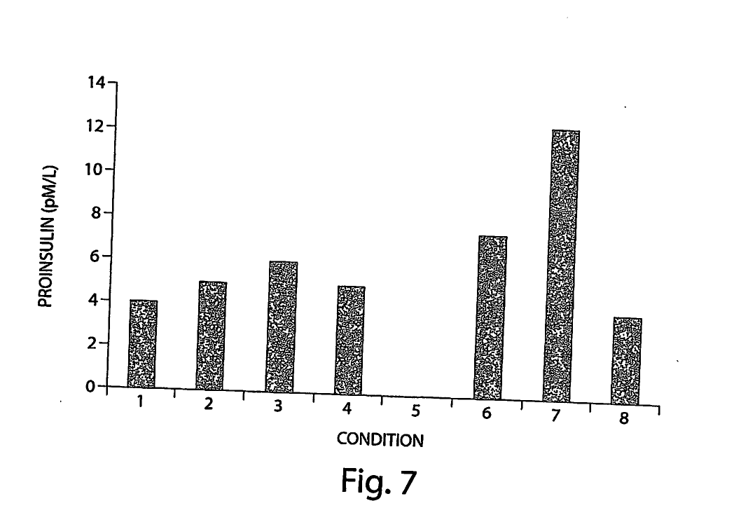

Cells cultured in the presence of retinoic acid alone tended to develop a higher level of insulin production than cell cultured in the presence of SHH and retinoic acid (FIG. 7).

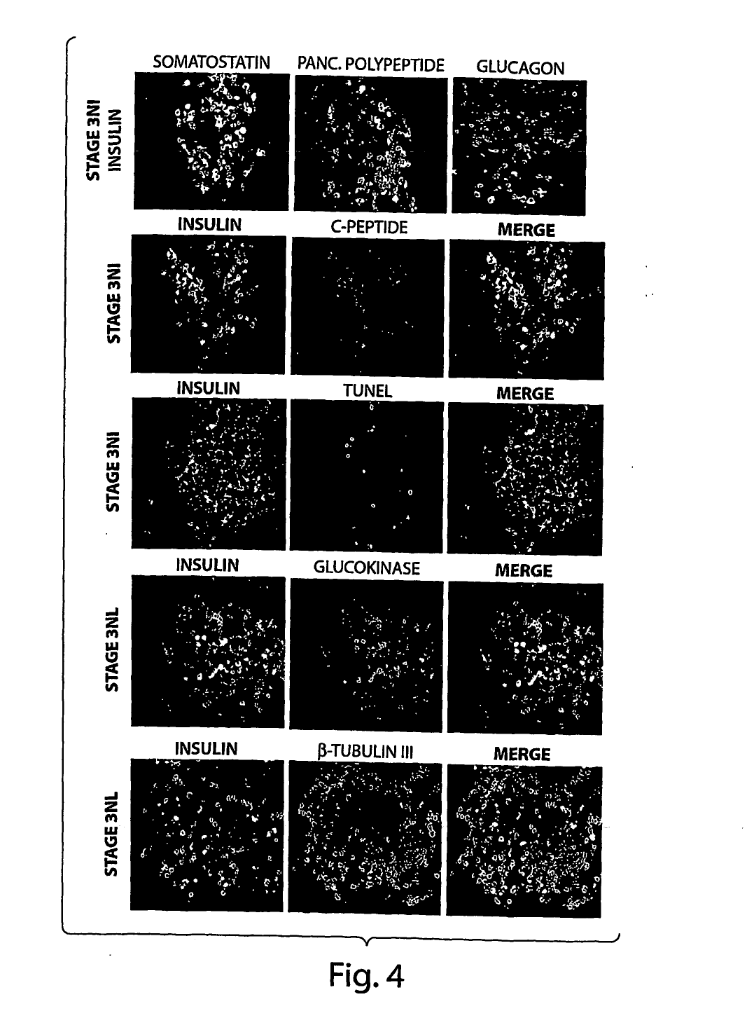

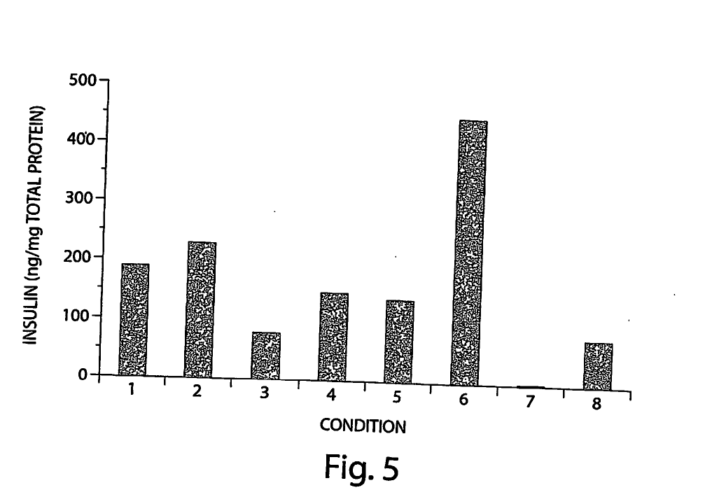

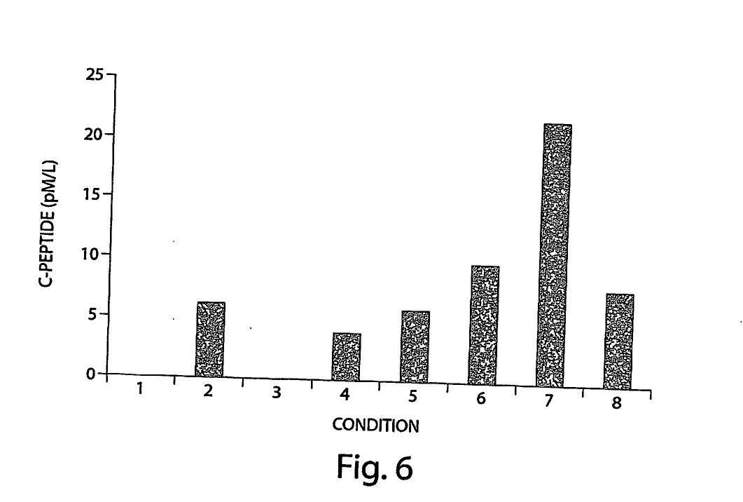

A variety of conditions were tested, and production of insulin, C-peptide and proinsulin was assessed (see FIGS. 4, 5 and 6 respectively). Conditions: (1) 100 nM Retinoic Acid+30 nM Sonic Hedgehog for 2 weeks then 10 mM Nicotinamide+2 nM Activin A for 1 week; (2) 200 nM Retinoic Acid for 2 weeks then 10 mM Nicotinamide+2 nM Activin A for 1 week; (3) 100 nM Retinoic Acid+30 nM Sonic Hedgehog for 2 weeks then 10 mM Nicotinamide+10 nM IGF-1 for 1 week; (4) 2000 nM Retinoic Acid for 2 weeks then 10 mM Nicotinamide+10 nM IGF-1 for 1 week; (5) 100 nM Retinoic Acid +30 nM Sonic Hedgehog for 2 weeks then 10 mM Nicotinamide+10 μM LY294002 for 1 week; (6) 200 nM Retinoic Acid for 2 weeks then 10 mM Nicotinamide+10 μM LY294002 for 1 week; (7) 100 nM Retinoic Acid+30 nM Sonic Hedgehog for 2 weeks then 10 mM Nicotinamide+1 mM Sodium butyrate for 1 week; (8) 200 nM Retinoic Acid for 2 weeks then 10 mM Nicotinamide+1 mM Sodium butyrate for 1 week.

3. Generation of Human Insulin Producing Cells

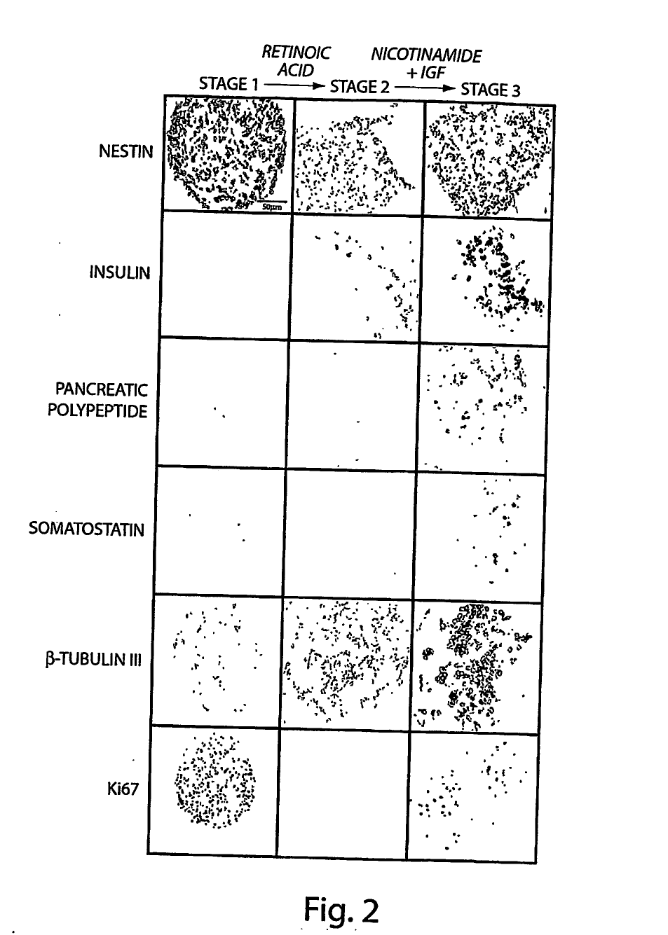

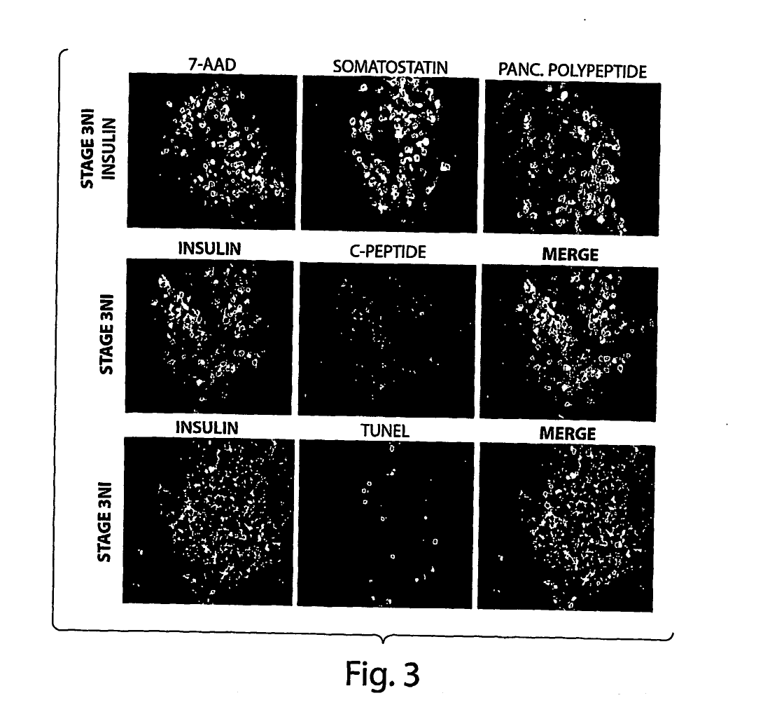

The methods described herein can be used to generate IPCCs efficiently from purified human NS cell lines as well as human embryonic stem cell lines and neuroendocrine stem cell lines. Undifferentiated NS cells (stage 1) uniformly express the marker nestin (FIG. 2) and the proliferation marker Ki67. Treatment of human NS cells with 1 micromolar retinoic acid for 6 days directed differentiation of IPCCs that produce 0.1% of insulin levels found in human pancreatic islets (stage 2) as measured by ELISA. Following treatment with retinoic acid, nestin expression is markedly reduced, and low levels of insulin expression in a subset of cells is detected (FIG. 2). Following treatment with nicotinamide and IGF-1 (stage 3 NI) for 7-10 days, nestin expression is nearly extinguished, while the number of insulin expressing cells increases. We find that 30-40% of cells in stage 3 clusters express insulin and C-peptide (FIG. 2). We also detected expression of glucagon and pancreatic polypeptide, two other hormones produced by islet cells. By stage 3, the majority of cells comprising IPCCs are not proliferating, as assessed by Ki67 expression (FIG. 2) Thus, the sequence of factor additions we have identified generates cells expressing several typical islet cell markers. Similar patterns of gene expression are observed following treatment of stage 2 IPCCs with nicotinamide and sodium butyrate (stage 3 NS; data not shown). Treatment of stage 2 IPCCs with nicotinamide and LY294002 resulted in 90% cell death, as assessed by TUNEL assay and immunohistochemical detection of activated caspase 3 (not shown). In contrast, nicotinamide and IGF-1 treatment during stage 3 NI produced IPCCs in which less than 5% of cells were apoptotic (FIG. 3). These experiments were performed with a NS line (#1651) obtained through Dr. Irving Weissman (Stanford Univ).

Apoptotic mouse ES cells in vitro can absorb significant amounts of bovine insulin which is routinely added to the culture medium. To examine if insulin we detected in our neurosphere studies was produced de novo, we measured expression and levels of insulin C-peptide and insulin messenger RNA. C-peptide is an internal region of the pre-proinsulin polypeptide chain that is removed during post-translational processing. Detection of human C-peptide provides evidence that insulin synthesis is occuring in human cell lines, because recombinant bovine insulin supplements used during cell culture lack C-peptide. Additionally, bovine insulin C-peptide has a primary sequence distinct from human C-peptide and does not cross react with specific human C-peptide antibodies used in our immunohistochemical or ELISA studies (FIG. 3).

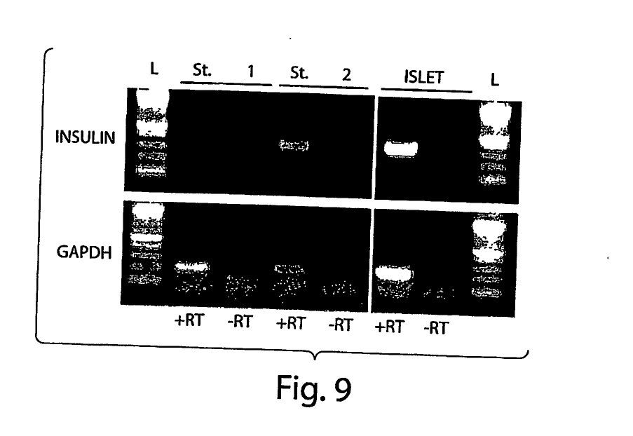

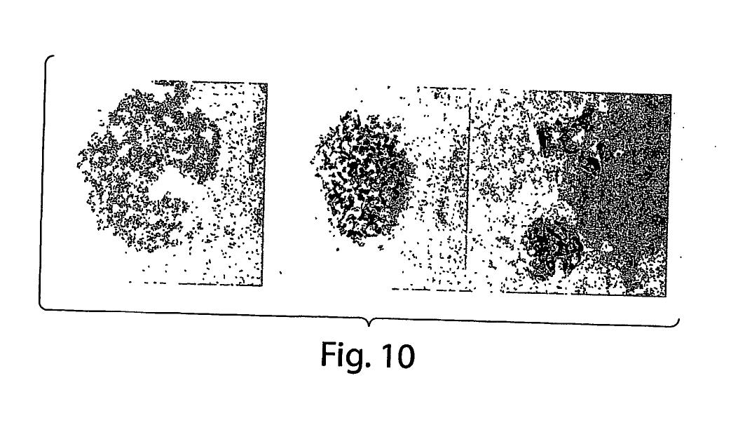

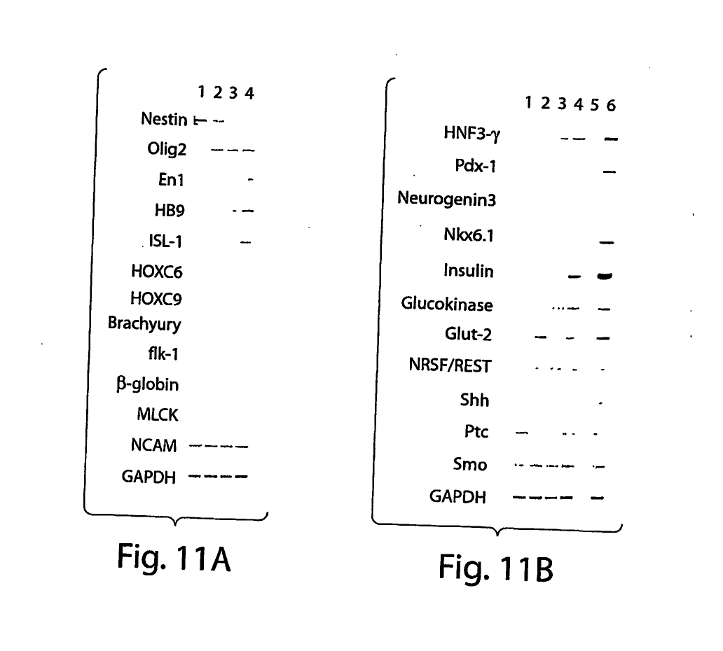

We detect human C-peptide in all insulin+ stage 3 IPCCs derived from human NS cells by immunohistochemistry (FIG. 3). Undifferentiated (stage 1) NS cells did not produce C-peptide as detected by ELISA studies. In stage 3 IPCCs we detected 0.2 nM C-peptide, approximately 0.5% of levels found in human islets . Insulin ELISA studies showed that insulin was present at 5-8% of levels found in human pancreatic islets. In islets, C-peptide and insulin are produced and secreted at equimolar concentrations, but in IPCCs we do not yet know if post-translational modification of preproinsulin results in equal concentrations of C-peptide and insulin. Thus, we conservatively estimate that levels of insulin production in NS cell-derived stage 3 IPCCs are approximately 0.5% of levels contained in human pancreatic islets. Detection of human insulin MRNA by RT-PCR (FIG. 9) and in situ hybridization methods (FIG. 10) in stage 2 and stage 3 human NS-derived IPCCs provides further evidence of insulin production resulting from our protocol. Similar results were obtained for detection of the Pdx1 mRNA (data not shown). Other RT-PCR studies indicate that a sequence of gene expression changes occur during IPCC development (FIG. 11). We find that neural stem cell markers are extinguished, whereas markers of endoderm like HNF3-gamma, and pancreatic cell types (like Pdx1 and insulin) are up-regulated. In contrast, we find little evidence of detectable mesodermal marker expression. Ongoing analysis of insulin and C-peptide synthesis by IPCCs includes detection and quantification by mass spectrometry, metabolic labelling, and ultrastructural studies of stage 3 IPCCs.

These data illustrate at least two independent strategies for generating an inexhaustable supply of human insulin-producing cell clusters (IPCCs). In the first strategy, human NS cells derived from fetal or adult sources, could be used to produce IPCCs. In the second strategy, human ES cells can be used to generate IPCCs.

4. In Vivo Function and Fate of Transplanted IPCCs

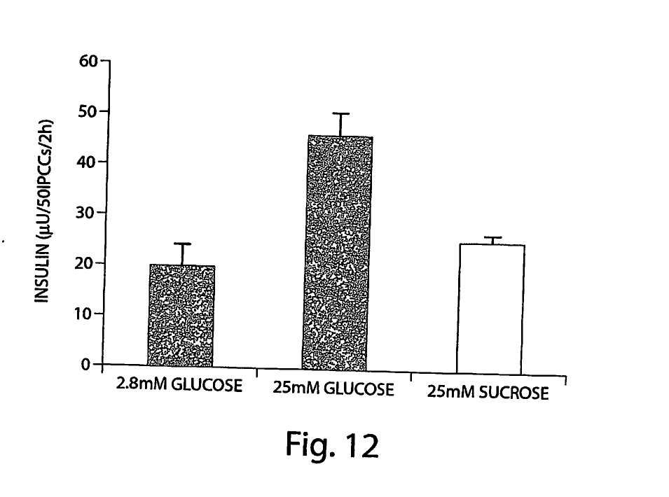

Our studies have shown that IPCCs derived from hNS or ES cells express factors crucial for regulating glucose sensing and insulin release in islet cells, including glucokinase, PDX1, and glucose transporters (data not shown). To determine if insulin-producing cell clusters derived from NS cells respond appropriately to glucose stimulation in vitro, we measure insulin release following stimulation with glucose (or osmotic controls like sucrose). As shown in FIG. 12, we observed insulin release by IPCCs following insulin challenge, suggesting that IPCCs are capable of responding to physiologically relevant stimuli to appropriately secrete insulin.

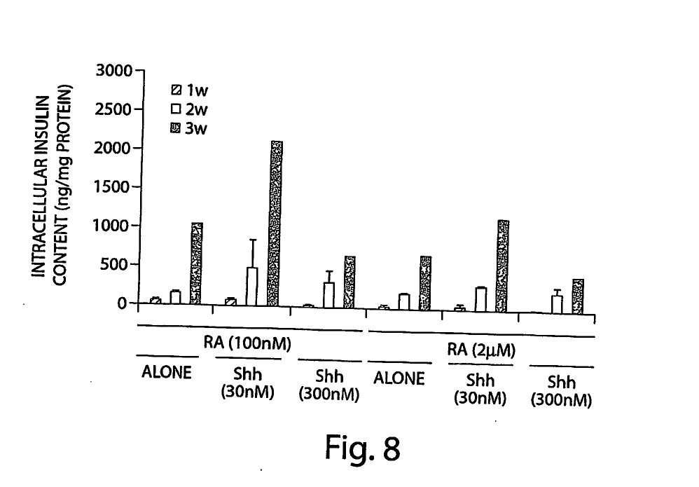

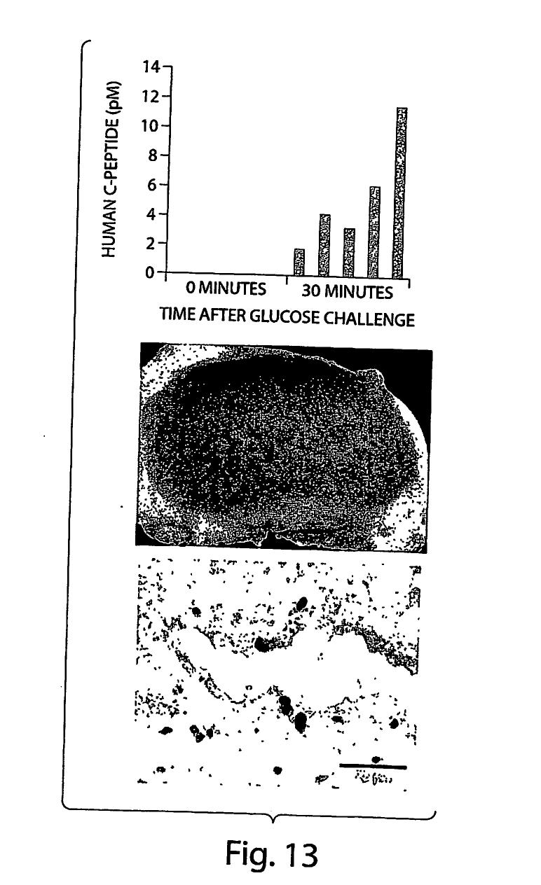

To determine if insulin-producing cell clusters derived from NS cells respond appropriately to glucose stimulation, we measured human C-peptide release from IPCCs transplanted into NOD-scid mouse recipients following glucose challenge. Initially, we transplanted five mice with 1000 stage 3 IPCCs, then challenged these animals at one and two weeks post-transplantation with intraperitoneal (IP) glucose challenge. As shown in FIG. 13, we reproducibly detected circulating human C-peptide in 5/5 mice at 30 minutes after IP glucose injection. No C-peptide was detected prior to injection or in 5 control mice that had received a sham transplantation. Following these studies, transplanted mice were sacrificed and grafts recovered for immunohistochemical analysis. As shown in FIG. 8, we detected ample amounts of C-peptide in cells recovered from IPCCs grafts at 3 weeks post-transplantation. Together, these data provide evidence that in vivo exposure of human NS cell-derived IPCCs to increased glucose resulted in release of the products of insulin synthesis. Thus, we postulate that IPCCs may be similar to islets in their responsiveness to appropriate stimuli promoting insulin secretion. To our knowledge, this is the first demonstration that hNS can be used to generate insulin-producing cells.

Incorporation by Reference

All publications and patents mentioned herein are hereby incorporated by reference in their entirety as if each individual publication or patent was specifically and individually indicated to be incorporated by reference. In case of conflict, the present disclosure, including any definitions herein, will control.

Equivalents

While specific embodiments of the subject disclosures have been discussed, the above specification is illustrative and not restrictive. Many variations of the disclosures will become apparent to those skilled in the art upon review of this specification and the claims below. The full scope of the disclosures should be determined by reference to the claims, along with their full scope of equivalents, and the specification, along with such variations.

1. An insulin-producing cell derived from a neural or neuroendocrine stem cell.

2. The insulin-producing cell of claim 1, wherein the neural or neuroendocrine stem cell is a cell from a neural or neuroendocrine stem cell line.

3. The insulin-producing cell of claim 1, wherein the insulin-producing cell is positive for one or more markers selected from the group consisting of: insulin C-peptide and glucokinase.

4. The insulin-producing cell of claim 1, wherein the insulin-producing cell does not produce glucagon, pancreatic polypeptide or somatostatin.

5. The insulin-producing cell of claim 1, wherein the insulin-producing cell is not apoptotic.

6. A cell cluster derived from neural or neuroendocrine stem cells, wherein the cell cluster comprises insulin-producing cells.

7. The cell cluster of claim 6, wherein at least 50% of the cells of the cell cluster comprise cytoplasmic insulin.

8. The cell cluster of claim 6, wherein the cell cluster further comprises at least one cell type selected from the group consisting of: glucagon producing cells, pancreatic polypeptide producing cells and somatostatin producing cells.

9. The cell cluster of claim 6, wherein at least 50% of the cells of the cell cluster are viable.

10. A method for making a cell composition comprising cells that are receptive to treatment with an islet cell differentiation factor, the method comprising culturing stem cells with a neural/endoderm caudalizing factor.

11. The method of claim 10, wherein the stem cells are neural or neuroendocrine stem cells.

12. The method of claim 11, wherein the stem cells are cells of a neural or neuroendocrine stem cell line.

13. The method of claim 10, wherein the cell composition comprises or is derived from a neural stem cell that is positive for binding to a monoclonal antibody AC133 or to a monoclonal antibody 5E12.

14. The method of claim 10, wherein the neural/endoderm caudalizing factor is caudalizing retinoic acid signaling activator.

15. The method of claim 14, wherein the caudalizing retinoic acid signaling activator is a retinoid.

16. The method of claim 14, wherein the neural/endoderm caudalizing factor is an all-trans retinoic acid or an ester, salt or free base thereof.

17. (canceled)

18. A method for producing insulin-producing cells, the method comprising:
a. culturing human stem cells with a neural/endoderm caudalizing factor to obtain a first cell composition;
b. culturing the first cell composition, or a portion thereof, with an islet cell differentiation factor, thereby obtaining a second cell composition comprising insulin-producing cells.

19. The method of claim 18, wherein the second cell composition additionally comprises one or more of the following cell types: somatostatin producing cells, pancreatic polypeptide producing cells and glucagon producing cells.

20. A cell composition comprising insulin-producing cells prepared according to the method of claim 18.

21. The method of claim 18, wherein at least 50% of the cells of the second cell composition are not apoptotic.

22. The method of claim 18, wherein culturing the first population of cells, or a portion thereof, with an islet cell differentiation factor comprises culturing the cells with nicotinamide.

23. The method of claim 22, wherein culturing the first population of cells, or a portion thereof, with an islet cell differentiation factor comprises culturing the cells with nicotinamide and an additional factor selected from the group consisting of IGF-1, AN IGF-1 AGONIST, a P13K inhibitor, butyric acid or a salt thereof, activin, GDF-8, GDF-11 and a hedgehog antagonist.

24-26. (canceled)

27. A method of ameliorating, in a subject, a condition related to insufficient pancreatic function, the method comprising administering to the subject an effective amount of insulin-producing cells produced according to the method of claim 18.

28. The method of claim 27, wherein the effective amount of insulin-producing cells causes an increase in blood insulin levels in the subject.

29. The method of claim 27, wherein the effective amount of insulin-producing cells causes an increased rate of glucose-induced insulin production in the subject.

30. The method of claim 27, wherein the subject has a diabetes caused by beta-cell insufficiency.

31-39. (canceled)

40. The method of claim 18, wherein the neural/endoderm caudalizing factor is selected from the group consisting of: a caudalizing retinoic acid signaling activator, a retinoid, and an all-trans retinoic acid or an ester, salt or free base thereof.

41-44. (canceled)

45. A method for ameliorating, in a subject, a condition related to insufficient pancreatic function, the method comprising:
a. obtaining from the subject or an HLA-matched donor a sample comprising neural or neuroendocrine stem cells;
b. culturing one or more of the neural or neuroendocrine stem cells in the presence of a neural/endoderm caudalizing factor to obtain a first cell composition;
c. culturing the first cell composition in the presence of an islet cell differentiation factor to obtain a second cell composition, wherein the second cell composition comprises insulin producing cells; and
d. administering to the subject an effective amount of insulin-producing cells.

46. The method of claim 45, wherein, prior to (b), the sample comprising neural or neuroendocrine stem cells is cultured so as to increase the number of neural or neuroendocrine stem cells.

47. The method of claim 45, wherein the sample is obtained from a tissue selected from the group consisting of: a tissue comprising cells of the peripheral nervous system, a tissue comprising cells of the central nervous system and a tissue comprising neuroendocrine cells.

48. The method of claim 45, wherein the sample is obtained by a method selected from among: trans-cranial biopsy, olfactory bulb biopsy, spinal cord biopsy and skin biopsy.

49. The method of claim 45, wherein the neural/endoderm caudalizing factor is a caudalizing retinoic acid signaling activator.

50. The method of claim 45, wherein the neural/endoderm caudalizing factor is a retinoid.

51. The method of claim 45, wherein the neural/endoderm caudalizing factor is is an all-trans retinoic acid or an ester, salt or free base thereof.

52. The method of claim 45, wherein culturing the first population of cells, or a portion thereof, with an islet cell differentiation factor comprises culturing the cells with nicotinamide.

53. The method of claim 45, wherein culturing the first population of cells, or a portion thereof, with an islet cell differentiation factor comprises culturing the cells with nicotinamide and an additional factor selected from the group consisting of IGF-1, AN IGF-1 AGONIST, a P13K inhibitor, butyric acid or a salt thereof, activin, GDF-8, GDF-11 and a hedgehog antagonist.
